(19) United States
(12) Patent Application Publication
(10) Pub. No.: US 2009/0227043 A1
(43) Pub. Date: Sep. 10, 2009

(54) Fluorescence Resonance Energy Transfer Assay Based on Modified Solid Surface

(76) Inventor: Wei Huang, Cupertino, CA (US)

Correspondence Address: KOLISCH HARTWELL, P.C., 200 PACIFIC BUILDING, 520 SW YAMHILL STREET, PORTLAND, OR, 97204, US

(21) Appl. No.: 12/389,289

(22) Filed: Feb. 19, 2009

Related U.S. Application Data
(60) Provisional application No. 61/066,363, filed on Feb. 19, 2008.
(60) Provisional application No. 61/133,857, filed on Jul. 2, 2008.

(51) Int. Cl. G01N 33/566

(52) U.S. Cl. 436/501

(57) ABSTRACT

System, including methods, apparatus, and kits, for fluorescence resonance transfer (FRET) binding assays that are surface-based.

### CROSS-REFERENCES TO PRIORITY APPLICATIONS

This application is based upon and claims the benefit under 35 U.S.C. § 119(e) of U.S. Provisional Patent Application Ser. No. 61/066,363, filed Feb. 19, 2008, and U.S. Provisional Patent Application Ser. No. 61/133,857, filed Jul. 2, 2008, each of which is incorporated herein by reference in its entirety for all purposes.

### INTRODUCTION

Assays may be performed to quantify one or a few analytes present in a biological sample, which is generally in aqueous form. In a typical “heterogeneous” assay, the analyte is separated from the rest of the sample before detection and quantification. Separation may involve a physical method, such as liquid chromatography or mass spectroscopy, or use of a binding partner that binds specifically to the analyte, to perform a binding assay.

Many types of biochemical molecules present in biological systems are naturally involved in molecular interactions with high specificity, and thus can function as binding partners in binding assays. Exemplary binding partners include proteins (e.g., antibodies, receptors, and enzymes) and nucleic acids (e.g., DNA/RNA molecules). Binding assays, such as clinical immunoassays, that use a binding partner of high specificity and affinity for an analyte, are designed to be simple and efficient. Binding assays that operate under the same principle also are used in environmental monitoring, biological research, and drug discovery.

Besides a binding partner, another key component of a binding assay is a detectable label (also termed a tracer) that can be measured to provide an indication of the amount of binding. Labels employed in binding assays include radioactive elements, chemically reactive molecules, chromogenic molecules and nanoscale molecular assemblies, inorganic catalysts, and enzymes, among others. Labels based on luminescence, including chemiluminescence and fluorescence, are finding wide usage due to several advantages associated with these labels, including sensitivity, simplicity, linear range, and low toxicity.

Luminescence is the emission of light from molecules in electronically excited states. Fluorescence is luminescence resulting from absorption of excitation light. The light emitted in fluorescence typically is at a lower energy or longer wavelength than the excitation light, with the difference between excitation and emission wavelengths termed the Stokes shift. A single fluorescent molecule can go through many cycles of excitation and emission to produce thousands of photons. This phenomenon, combined with the advance of technologies such as photomultiplier tubes (PMT) for the sensitive detection of photons, contributes to the high sensitivity associated with fluorescence technologies.

The use of fluorescence technologies enables novel “homogeneous” assays, also called “mix and read” assays, which do not require separation or washing steps. In homogeneous assays, binding generally takes place in the solution (fluid) phase, and the binding produces an alteration in the photochemical properties of a label that can be measured directly from the solution phase. For example, homogeneous binding assays based on fluorescence resonance energy transfer (FRET) between labels have found many applications in biological studies. In particular, FRET is a popular assay technology used in high throughput screening (HTS) applications for drug discovery.

High throughput screening is one of the main engines in modern drug discovery. This type of screening involves testing a large collection of samples in a biochemical or cell-based assay to identify potential lead compounds that can serve as a starting point for further optimization. The samples often contain individual, small-molecule compounds that have been accumulated for years in pharmaceutical companies or synthesized using combinatorial chemistry. The samples also may contain a collection of processed natural products. The number of compounds in a collection, also called a compound library, may range from a few thousand to millions. Screening of a large number of compounds is enabled by the use of high density (e.g., 384-well or 1536-well) microplates, automation in liquid handling, and advances in assay technologies. Because automation is essential for increasing the throughput of screening, homogeneous assays are usually preferred for high throughput screening applications. Among the homogeneous assay technologies used in high throughput screening applications, a time-resolved FRET assay may be the most commonly used due to the sensitivity, flexibility, and simplicity of this technology.

FRET occurs when the emission spectrum of a fluorescent molecule, an energy donor, overlaps with the absorption spectrum of another molecule, an energy acceptor. When the donor is excited, FRET results in reduction of the intensity of donor emission, as energy from the donor in its excited state is transferred to the acceptor. The acceptor can be either fluorescent or non-fluorescent. If the acceptor is fluorescent, the intensity of acceptor emission is increased as a result of FRET. A second critical criterion of energy transfer in FRET is close proximity between energy donor and acceptor. The efficiency of energy transfer in FRET decreases with or nearly to the 6th power of the distance between the donor and acceptor. A principal factor in the strong dependence of FRET on distance is believed to be that energy transfer between donor and acceptor requires a coupled dipole-dipole interaction, wherein transfer occurs when the dipole orientations are aligned, with no intermediate photon involved. Regardless of mechanism, the distance between donor and acceptor molecules is a dominant factor in determining the extent of energy transfer, with energy transfer dropping off precipitously as the distance is increased beyond the optimal separation.

The principle of a typical homogeneous FRET system 40, with an assay performed in an assay solution 42, is illustrated in FIG. 1. First and second binding partners 44, 46 (e.g., antibody and antigen) are labeled, respectively, with a first member 48 and a second member 50 of a FRET pair, namely, an energy donor and energy acceptor (e.g., a pair of fluorophores).

In the absence of binding, the donor and the acceptor are distributed independently in assay solution 42. As a result, the average distance between the donor and acceptor is much larger than that required for efficient fluorescence resonance energy transfer, indicated at 52. This critical distance is called the “Förster distance” and is typically in the range of 30-100 Å, depending on the FRET pair involved in FRET 52.

When binding partners 44, 46 bind to one another and form a binding complex 54, the average distance between FRET members 48, 50 is drastically reduced to place the FRET members in close proximity, and the efficiency of FRET 52 is greatly enhanced. As a result of binding, when the assay solution is illuminated at the excitation wavelength of the donor, fluorescence emission of the acceptor increases, relative to no binding, while fluorescence emission of the donor decreases.

The assay shown in FIG. 1 is a “homogeneous” assay because the entire assay takes place in the solution phase, and there is no washing step required for separation of bound from unbound components. Furthermore, this assay may be a “time-resolved” or “time-gated” FRET assay if illumination with excitation light and detection of emission light are performed at nonoverlapping times, typically with a time delay between the end of illumination and the start of detection.

Homogeneous FRET assays provide the “mix and read” simplicity that is critical for laboratory automation, particularly for high throughput screening applications. Nevertheless, there is a common disadvantage in current homogenous FRET assays caused by the capacity of substances in the assay solution to absorb light (i.e., act as chromophores). This absorption of light may produce “inner filter effects” that may (1) reduce the intensity or alter the spectrum of excitation light that reaches the energy donor, (2) produce nonuniform excitation of the energy donor within the assay solution, and/or (3) reduce the intensity or alter the spectrum of emitted light that reaches the detector. These inner filter effects may be caused by any substances in the assay solution that absorb light at the wavelength of excitation or emission.

Inner filter effects may cause at least two adverse consequences to assays. First, the presence of interfering substances often varies among samples, independent of the target analyte(s) of the assay, resulting in a sufficiently high background variability in the detected assay signal that a homogeneous FRET assay is rendered inaccurate and/or unreliable in some applications. Second, and independent of sample-to-sample variability, inner filter effects may decrease the signal-to-noise ratio of FRET assays, thereby reducing the sensitivity, reliability, and/or utility of specific FRET partners favorable to particular assay methodologies (e.g., time-resolved FRET) and/or sample applications (e.g., tissue culture medium). Thus, there is a need for improved FRET assays that preserve the “mix and read” simplicity of conventional homogeneous FRET assays while minimizing optical interference caused by light-absorbing substances in the assay solution.

### SUMMARY

The present disclosure provides a system, including methods, apparatus, and kits, for fluorescence resonance transfer (FRET) binding assays that are surface-based.

### BRIEF DESCRIPTION OF THE DRAWINGS

FIG. 1 is a schematic view of a conventional system for performing FRET binding assays in a homogeneous assay solution.

FIG. 2 is a schematic view of selected aspects of an exemplary system for performing FRET binding assays that are surface-based, in accordance with aspects of the present disclosure.

FIG. 3 is a schematic flowchart illustrating an exemplary method of performing FRET binding assays that are surface-based, in accordance with aspects of the present disclosure.

FIGS. 4-7 are schematic flowcharts illustrating exemplary approaches for connecting a member of a FRET pair and a binding partner to the surface of a solid support in the method of FIG. 3, in accordance with aspects of the present disclosure.

FIG. 8 is a schematic view of selected aspects of another exemplary system for performing FRET binding assays that are surface-based, with the system including a FRET-capable binding complex formed with an unlabeled analyte in a “sandwich” configuration, in accordance with aspects of the present disclosure.

FIG. 9 is a schematic view of selected aspects of yet another exemplary system for performing FRET binding assays that are surface-based, with the system including an unlabeled analyte that inhibits formation of a binding complex by competing with a labeled analyte analogue for a limiting number of binding sites, in accordance with aspects of the present disclosure.

FIG. 10 is a schematic view of selected aspects of still another exemplary system for performing FRET binding assays that are surface-based, with the system including a flexible linker that tethers a second member of a binding pair and a second member of a FRET pair to a solid support, in accordance with aspects of the present disclosure.

FIG. 11 is an isometric view of an exemplary microplate that provides a plurality of spaced solid supports in a system for performing FRET binding assays that are surface-based, in accordance with aspects of the present disclosure.

FIG. 12 is a cross-sectional view of a well of the microplate of FIG. 11, taken generally along line 12-12 of FIG. 11 through the well and one of the solid supports (the bottom wall of the well), in accordance with aspects of the present disclosure.

FIG. 13 is a schematic view of selected aspects of an exemplary system for performing surface-based FRET binding assays using an optical fiber as a solid support, in accordance with aspects of the present disclosure.

FIG. 14 is a view of the system of FIG. 13, taken generally at “14” in FIG. 13 around an end of the optical fiber that provides a support surface for binding.

FIG. 15 is a graph of fluorescence intensity measured after treatment of a 96-well, clear acrylic microplate with a europium (Eu) chelate solution for different periods of time, in accordance with aspects of the present disclosure.

FIG. 16 is a bar graph of FRET signals measured from binding of streptavidin-allophycocyanin (APC) in solution to surface-connected biotin in microplate wells, with the biotin connected, via bovine serum albumin (BSA), to a well surface that has been modified with an exemplary lanthanide, namely, a europium chelate, in accordance with aspects of the present disclosure.

FIG. 17 is a bar graph of FRET signals measured generally as in FIG. 16 for wells with surface-connected biotin-BSA, but with the signals read from both the top and the bottom of the wells, and with and without of an exemplary colored compound, phenol red, in the assay solution to provide simulated interference, in accordance with aspects of the present disclosure.

FIG. 18 is a bar graph of log fluorescence intensity measured after treatment of wells of a 384-well polystyrene plate with a europium chelate in either DMSO or xylene, in accordance with aspects of the present disclosure.

FIG. 19 is a bar graph of log fluorescence intensity measured after treatment of wells of a 384-well polystyrene plate with different concentrations of a europium chelate (Eu-NTA) dissolved in DMSO, in accordance with aspects of the present disclosure.

FIG. 20 is a bar graph of FRET signals measured from binding of streptavidin-APC in solution to surface-connected biotin in microplate wells, with the well surface to which biotin is connected modified by treatment with the highest concentration of europium chelate from FIG. 19, with FRET signals read from the top and from the bottom of the microplate wells, with and without free biotin (400 μM biotin) as a competitor, and with and without simulated interference from an exemplary colored compound, phenol red, in the assay solution, in accordance with aspects of the present disclosure.

FIG. 21 is a table of FRET signals measured in binding assays performed with microplate wells that have been surface-modified by treatment with different concentrations of biotin-APC and in the presence of different concentrations of Eu-streptavidin in the assay solution, in accordance with aspects of the present disclosure.

FIG. 22 is a bar graph of FRET signals measured in binding assays performed in microplate wells with 1 nM Eu-streptavidin in the assay solution and with the different concentrations of biotin-APC for surface modification of microplate wells used in FIG. 21.

FIG. 23 is a bar graph of fluorescence intensity measured from wells of a 384-well microplate treated with different concentrations of a Cy5 solution in DMSO, in accordance with aspects of the present disclosure.

FIG. 24 is a bar graph of FRET signals measured from binding of Eu-streptavidin in solution to surface-connected biotin-BSA using wells of a 384-well microplate that are also surface-modified with Cy5 as in FIG. 23, in accordance with aspects of the present disclosure.

FIG. 25 is a table of FRET signals measured from wells of a 384-well microplate treated with biotin-APC in a gelatin solution to connect the biotin-APC to the wells by entrapment in a gelatin matrix before contact with different concentrations of Eu-streptavidin in solution, in accordance with aspects of the present disclosure.

FIG. 26 is a schematic view of selected aspects of another exemplary system for performing FRET binding assays that are surface-based, with the system including a film of inorganic lanthanide connected to a solid support to provide an energy donor of a FRET pair, in accordance with aspects of the present disclosure.

FIG. 27 is a bar graph of FRET signals measured from wells of a microplate assayed for binding of streptavidin-APC in solution to surface-connected biotin-BSA using well surfaces modified with a europium chelate in aqueous solution, in accordance with aspects of the present disclosure.

FIG. 28 is a bar graph of FRET signals measured from wells of a microplate assayed for binding of biotin-allophycocyanin (biotin-APC) in solution to surface-connected Eu-streptavidin, in accordance with aspects of the present disclosure.

FIG. 29 is a bar graph of FRET signals measured from wells of a microplate assayed for streptavidin-allophycocyanin binding to biotin-BSA on well surfaces to which a europium chelate is chemically bonded, in accordance with aspects of the present disclosure.

FIG. 30 is a schematic view of selected aspects of still yet another exemplary system for performing FRET binding assays that are surface-based, with the system including a matrix formed as a scaffold on a solid support and including a long-chain polymer that is connected to and supports a FRET member and a binding partner, in accordance with aspects of the present disclosure.

FIG. 31 is a bar graph of FRET signals measured from wells of a microplate assayed for binding of biotin-APC in solution to streptavidin, with the streptavidin and a europium chelate being connected to a matrix, which in turn is connected to surfaces of the wells, in accordance with aspects of the present disclosure.

FIG. 32 is a bar graph of FRET signals measured from wells of a microplate assayed for binding of biotin-APC in solution to surface-connected streptavidin generally as in FIG. 31 except with the use of a two-step coupling procedure, in accordance with aspects of the present disclosure.

### DETAILED DESCRIPTION

The present disclosure provides a system, including methods, apparatus, and kits, for fluorescence resonance transfer (FRET) binding assays that are surface-based. In some embodiments, the system may improve upon conventional homogeneous binding assays of samples based on FRET, by minimizing optical interference from sample components while preserving the “mix and read” characteristics of conventional FRET assays.

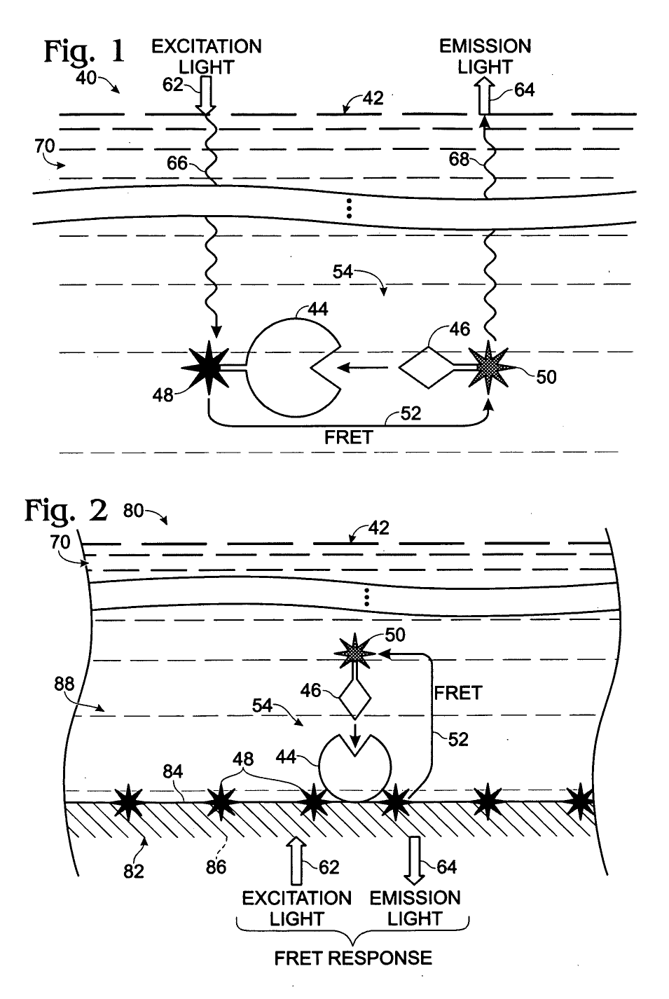

FIGS. 1 and 2 show respective, schematic views of a conventional, homogeneous FRET assay system 40 (FIG. 1) and an exemplary system for performing FRET binding assays that are surface-based (FIG. 2). Comparison of conventional FRET system 40 and the surface-based FRET system of FIG. 2 illustrate how the surface-based system of the present disclosure can reduce optical interference.

In conventional FRET system 40, excitation light 62 and emission light 64 must travel on excitation and emission paths 66 and 68 through a substantial portion 70 of assay solution 42 to permit measurement of FRET from a binding complex 54. The binding complex is distributed uniformly throughout the assay solution. Accordingly, both the excitation light and the emission light may travel, on average, halfway through the assay solution in order to reach the binding complex (excitation light) and then the detector (emission light). As a result, any light-absorbing substances positioned along paths 66, 68 may affect FRET signals measured in the assay, thereby affecting the accuracy, sensitivity, and/or reliability of the conventional FRET system.

FIG. 2 shows a surface-based FRET system 80 that obviates problems associated with light absorption throughout the assay solution in conventional FRET system 40. System 80 includes a solid support 82 having first and second surfaces 84, 86. Here and in many of the other figures, the solid support is shown with an indeterminate opposing second surface because the second surface generally would be relatively far from the first surface at the schematic molecular scale used to illustrate the assay system.

First binding partner 44 and first FRET member 48 (donor or acceptor) may be connected to first surface 84, to substantially immobilize binding partner 44 and FRET member 48 near and/or at the first surface. Accordingly, when first binding partner 44 interacts with second binding partner 46, a binding complex 54 connected to first surface 84 is formed, with the binding complex disposed in a minor or restricted portion 88 of the assay solution, which generally represents only a very tiny fraction of the overall volume of the assay solution. The binding complex thus places FRET members 48, 50 near one another for efficient energy transfer and near first surface 84, sequestered from the bulk of assay solution 42. This sequestration permits excitation light 62 and emission light 64 to travel, respectively, to and from FRET members 48, 50, via solid support 82, while avoiding the bulk of the assay solution, since binding complex 54 is concentrated near solid support 82. Stated differently, the excitation and emission light do not need to travel through a substantial portion of the assay solution and therefore fluorescence detection is not influenced substantially by substances in the assay solution that would normally cause interference in conventional FRET binding assays. Instead, illumination with excitation light and detection of emission light are conducted from a side of the solid phase surface that is opposite from an opposing side that contacts the assay solution. Therefore, optical interference from light-absorbing substances in the assay solution is minimized, which may provide better accuracy, sensitivity, and reliability than the conventional FRET system of FIG. 1.

The system may provide a method of detecting an analyte in a fluid sample. A binding partner may be contacted with a fluid sample, with the binding partner connected to a first surface of a solid support that also has a first FRET member of a fluorescence resonance energy transfer (FRET) pair connected to the first surface. A binding complex may be formed, with the binding complex connected to the first surface via the binding partner and including a second member of the FRET pair. Formation of the binding complex may be affected by an analyte, if any, in the fluid sample. The FRET pair may be exposed to excitation light via a second surface of the solid support, with the second surface spaced from the sample. A FRET response of the FRET pair to the step of exposing may be measured by detecting emitted light received from the second surface. Excitation light may reach the FRET pair, and emitted light may be received from the FRET pair, without passing through a substantial portion of the sample, thereby minimizing optical interference from the sample. The FRET response may be correlated with an amount of the analyte in the sample.

The system may provide another method of detecting an analyte in a fluid sample. A binding partner may be added to a fluid sample, with the binding partner connected to a member of a fluorescence resonance energy transfer (FRET) pair. The surface of a solid support may be contacted with the fluid sample. The solid support may be substantially transparent and may have another member of the FRET pair substantially immobilized with respect to the surface. A binding complex may be formed, with the binding complex connected to the surface via the binding partner. Excitation light may be passed through the solid support towards the surface to expose the FRET pair to the excitation light. A FRET response of the FRET pair to exposing may be measured by detecting emitted light that has traveled through the solid support away from the surface. Excitation light may reach the FRET pair, and emitted light may be received from the FRET pair, without passing through a substantial portion of the sample, thereby minimizing optical interference from the sample. The FRET response may be correlated with an amount of the analyte in the sample.

The system may include a device for assaying an analyte in fluid samples. The device may comprise a microplate forming a plurality of wells for holding fluid samples and each having a bottom wall that is substantially transparent such that optical detection can be performed from below the bottom wall. The bottom wall may include upper and lower surfaces. The device also may comprise a fluorescent lanthanide connected to the upper surface of each bottom wall, with about a same amount of the fluorescent lanthanide being connected to each upper surface, thereby providing a similar environment in each of the wells for surface-based fluorescence resonance energy transfer (FRET) assay of fluid samples.

The surface-based FRET binding assays disclosed herein may offer one or more substantial advantages over current solution phase, time resolved FRET technologies based on lanthanide chelates. These advantages may include any combination of the following: (1) reduced interference from inner filter effects because light passes through a constant solid phase instead of through an assay solution, (2) reduced interference from chelating agents, such as EDTA, in the assay solution, which may destabilize a fluorescent chelate, (3) the number of fluorophore molecules (or quencher molecules) associated with each biomolecule may not always be limited by the number of available functional groups on the biomolecule, (4) the potential for use of inorganic lanthanide phosphors with various fluorescence properties, (5) the potential for multiplexing assays based on spatial separation of components for distinct assays on a support surface and/or spectral separation of different lanthanide phosphors (i.e., optically distinguishable energy donors (and/or acceptors)), and (6) a higher fluorescence quantum yield, among others.

Further aspects of the present disclosure are provided by the following sections: (I) definitions, (II) assay methods, (III) connection of assay components to a solid support, (IV) assay configurations, (V) solid supports, (VI) kits, (VII) examples.

### I. DEFINITIONS

“Fluorescence resonance energy transfer,” also termed “Förster resonance energy transfer” and abbreviated as “FRET,” generally comprises an energy transfer that occurs between two chromophores, namely, an energy donor (a fluorophore) and an energy acceptor (optionally a fluorophore), as a result of absorption of excitation light by the energy donor. Although the present disclosure should not be limited to any theory of how FRET occurs, the energy transfer may be through a coupled dipole-dipole interaction and may be a nonradiative transfer from donor to acceptor, without generation of an intermediate photon. The efficiency of energy transfer may be strongly dependent on the separation distance between the donor and acceptor, such as varying by an inverse sixth power law. Accordingly, most FRET, for practical purposes, may be limited to a separation distance of less than about ten nanometers, such as about 30 to 100 øngströms. Also, the efficiency of energy transfer may be dependent on the spectral overlap of donor emission and acceptor absorption. In any event, the donor may be described as a fluorescent dye or a fluorophore, which may fluoresce in response to excitation by excitation light, and the acceptor also may be described as a fluorescent dye or a fluorophore, which may fluoresce in response to energy transfer from the donor, or may be described as a substantially non-fluorescent quencher of donor fluorescence.

A “FRET member” or a “member of a FRET pair” generally comprises an energy donor or an energy acceptor of a donor-acceptor pair capable of FRET when in close proximity and with exposure to excitation light of a suitable wavelength. Accordingly, members of a FRET pair generally are or include a fluorophore (the donor) having an emission spectrum that overlaps the absorption spectrum of a chromophore (the acceptor). The donor-acceptor pair may be described as a “FRET pair” of first and second FRET members, with the designations “first” and “second” referring respectively to the donor and the acceptor or respectively to the acceptor and the donor. Exemplary FRET pairs may include fluorescein/rhodamine, Cy3/Cy5, lanthanide/phycobiliprotein, lanthanide/Cy5, and cyan fluorescent protein (CFP)/yellow fluorescent protein (YFP), among others.

A “lanthanide,” which also may be called a “lanthanoid,” generally comprises any substance that includes one or more rare earth elements of atomic numbers 57 to 71, such as lanthanum, europium, and/or terbium, among others. The lanthanide may be an inorganic substance or an organic substance. If the lanthanide is an inorganic substance, the lanthanide may be described as an inorganic phosphor. Alternatively, the lanthanide may be a lanthanide chelate produced by chelation of at least one lanthanide ion with a chelator to form a lanthanide chelate. The chelator may be an organic compound and generally functions as a bi- or multidentate ligand for each lanthanide ion to form a chelate complex. Exemplary chelates may include cryptates, among others.

Lanthanide chelates may be used as energy donors in the FRET assays disclosed herein. In some embodiments, these chelates may provide substantial sensitivity, particularly in time-resolved FRET assays, due to their relatively long fluorescence lifetime, large Stokes shift, and/or sharp emission peaks. Among lanthanides, europium and/or terbium chelates, for example, may be suitable. In some embodiments, the excitation wavelength of a europium chelate may be in the UV range (e.g., 340 nm) and/or the emission wavelength may be in the red region of the spectrum (e.g., 615 nm).

A “phycobiliprotein” generally comprises a protein that is a member of the family of pigments including allophycocyanin, phycocyanin, phycoerythrin, and phycoerythrocyanin. A phycobiliprotein, such as allophycocyanin (APC), may function as a suitable acceptor for a donor, for example, a lanthanide, such as a lanthanide chelate like a europium chelate. Allophycocyanin may have a broad absorption spectrum and an emission peak at around 660 nm. APC may be an intensely fluorescent protein from blue-green algae and/or red algae. The molecular weight of APC appears to be approximately 100 kD and each protein molecule apparently contains approximately six bilins, which may be open-chain tetrapyrrole groups covalently bound to the protein and which appear to be responsible for the fluorescence of the protein.

A “FRET response,” which may be described as a “FRET signal,” generally comprises any measurable signal(s) indicative of FRET. The FRET response may be measured in any suitable manner, including illumination with excitation light, detection of one or more emission signals, and performance of mathematical operations on the one or more emission signals. For example, if the FRET acceptor is fluorescent, the FRET response may be measured as an increase in emission intensity from the acceptor at a wavelength (or wavelength range) of acceptor emission. Alternatively, or in addition, the intensity of light emission from both donor and acceptor may be measured and the FRET response may, for example, be expressed as the ratio of the intensities, optionally multiplied by an arbitrary number. For example, if the arbitrary number is selected to be 1000, the FRET response may be expressed as follows:

FRET response=1000*acceptor emission intensity/donor emission intensity

If a donor with a long fluorescence lifetime is used, a time resolved (time-gated) fluorescence detection method may be used. In this case, there is a delay between the excitation of the donor fluorophore and detection of emitted light.

A “binding partner,” which may be described as a “specific binding partner” or a “member of a specific binding pair,” generally comprises any member of a pair of binding members that bind to each other with substantial affinity and specificity. A pair of binding partners may bind to one another to the substantial exclusion of at least most or at least substantially all other components of a sample, and/or may have a dissociation constant of less than about 10⁻⁴, 10⁻⁶, or 10⁻⁸ M, among others. A pair of binding partners may “fit” together in a predefined manner that relies on a plurality of atomic interactions to cooperatively increase specificity and affinity. Binding partners may be derived from biological systems (e.g., receptor-ligand interactions), chemical interactions, and/or by molecular imprinting technology, among others. Exemplary corresponding pairs of binding partners, also termed specific binding pairs, are presented in the following table, with the designations “first” and “second” being arbitrary and interchangeable:

| First Binding Partner | Second Binding Partner |
|---|---|
| Antibody | Antigen |
| Receptor | Ligand |
| Biotin | Avidin/Streptavidin |
| Enzyme | Substrate/Inhibitor |
| Sense Nucleic Acid (e.g., | Antisense Nucleic Acid (e.g., |
| DNA/RNA) | DNA/RNA) |
| Trivalent Metal Ion (e.g., | Phosphate (e.g., |
| Ga³⁺) | phosphorylated peptide |
| Aptamer (e.g., nucleic acid or | Target |
| peptide) | |

Each binding partner may be an unlabeled binding partner, which is not connected to a FRET member, or may be a labeled binding partner that is connected to a FRET member, such as covalently or by a binding partner interaction.

A “binding complex” generally comprises a complex formed by binding of a corresponding pair of binding partners to one another. Accordingly, the binding complex may be formed with substantial specificity and affinity, and generally results in noncovalent association of the binding partners with one another.

The term “connected” generally characterizes direct attachment of a member (e.g., a FRET member, binding partner, matrix, solid support, surface, etc.) of a FRET assay system to another member of the system, or indirect attachment of the members via a bridge (e.g., a solid support, a matrix, a linker, and/or the like). The attachment may be covalent, which may be through a chemical bond or an uninterrupted series of two or more chemical bonds (and intervening one or more atoms) extending, collectively, from one member to the other. Accordingly, members may be connected covalently by formation of at least one chemical bond between the members. Alternatively, the attachment may, at least in part, be through (a) a binding interaction of a corresponding pair of binding partners each included in a member or disposed generally between the members, (b) nonspecific interactions between the members themselves or at least one or both of the members and a bridge (e.g., protein adsorption to a solid support surface), and/or (c) entrapment of one of the members within the other member. Connection of a member(s) (e.g., a FRET member and/or a binding member) to the surface of a solid support may form a modified surface of the solid support. Furthermore, a member connected to the first surface of a solid support having first and second surfaces is disposed closer to the first surface than the second surface.

A connected member also or alternatively may be described as being substantially immobilized with respect to another member, which means that the connected member is restricted from relative movement on a macroscopic scale with respect to the other member, such movement that would completely separate the connected member from the other member.

Various strategies may be utilized to connect a binding partner to the surface of a solid support. For example, the binding partner can be immobilized directly through formation of a chemical bond between the surface and the binding partner, e.g., an amino group on a protein such as an antibody can be linked to a carboxyl group on a polymer surface. In other cases, the binding partner alternatively can be adsorbed to a solid phase surface through noncovalent interactions. Alternatively, or in addition, the binding partner may be immobilized with respect to the surface indirectly. For example, a binding partner modified with biotin can be immobilized on a surface modified with streptavidin through a biotin/streptavidin interaction. Other binding partners that can be used for indirect immobilization include proteins that bind to antibodies (e.g., protein A and protein G) and secondary antibodies (e.g., anti-mouse antibody). For DNA or RNA, other DNA or RNA molecules can be used in indirect immobilization.

A “surface” generally comprises an external interface, or portion thereof, of a solid support or other solid phase structure. A surface therefore may be a surface region that constitutes only a subset of the entire external interface of a solid support or other solid phase structure. The external interface may be in contact with fluid (liquid and/or gas) and/or with another discrete solid phase structure.

An “analyte” generally comprises any substance undergoing analysis in a FRET binding assay, and may be termed a “substance of interest.” The analyte may be analyzed to determine its amount (e.g., presence/absence, concentration, quantity (such as number of moles/molecules), relative increase/decrease in concentration or quantity, relationship to a threshold level, etc.) in a sample. Alternatively, or in addition, the analyte may be analyzed to determine an activity (e.g., inhibition/activation of a chemical reaction, such as an enzyme-catalyzed reaction) of another analyte in a sample. The other analyte may be one or more members of a library of compounds being screened in a plurality of samples. Exemplary analytes may include inorganic substances, small organic molecules, biopolymers, or the like. Accordingly, analytes may include inorganic ions, nucleic acids (monomers or polymers), amino acids, peptides, proteins, lipids, carbohydrates, or the like. In some embodiments, the analyte may be a reactant or a product of a chemical reaction performed in a sample/assay solution. The chemical reaction may be catalyzed by an enzyme in the sample and thus the analyte may be a substrate or a product of an enzyme-catalyzed reaction performed in the sample.

Terms describing molecular-scale members of an assay system, such as “a FRET member,” “a FRET pair,” “a binding partner,” “a binding complex,” and “a chain,” are used in the singular in the present disclosure for clarity and simplification. However, each term may represent many individual moieties, molecules, and/or complexes, of the molecular-scale member.

### II. ASSAY METHODS

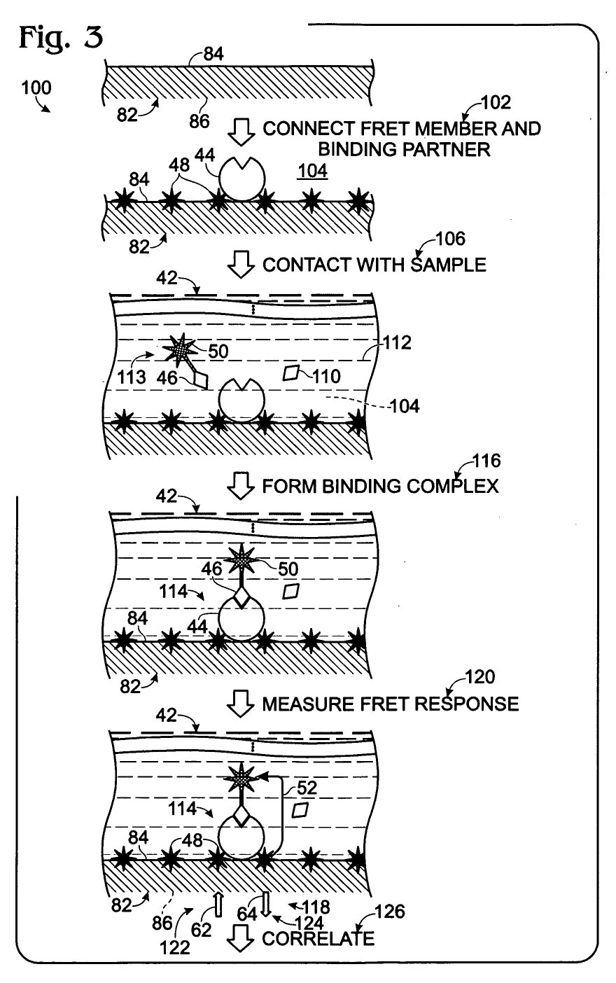

This section provides exemplary FRET assay methods that may be performed according to the present disclosure. The steps described below in relation to a method 100 of FIG. 3, and for methods elsewhere in the present disclosure, may be performed in any suitable combination, in any suitable order, and any suitable number of times.

Solid support 82 may be selected. The solid support may be configured as a sample holder for supporting and/or containing any suitable number of samples in any number of isolated compartments. The solid support may include first surface 84 and second surface 86, which may be spaced from one another and/or at least generally opposing, such as surfaces that are substantially parallel, as shown here, or oriented obliquely or substantially orthogonally to one another, among others. Each surface may be substantially planar or nonplanar, as appropriate.

First binding partner 44 and/or first FRET member 48 (an energy donor or an energy acceptor of a FRET pair) may be connected, indicated at 102, to first surface 84 of solid support 82. The first binding partner and/or first FRET member may be described as being “pre-connected,” which means connection before contact with a sample/assay solution. The binding partner and FRET member may be connected serially or in parallel and may be distributed independently or may have corresponding distributions. Furthermore, the first binding partner and first FRET member may (or may not) be connected to one another, covalently or noncovalently (e.g., by binding partners), before they are connected to the solid support. Each of the first binding partner and the FRET member may be disposed below and/or above first surface 84, for example, within solid support 82, or disposed at and/or above the surface in contact with a void 104 for receiving sample adjacent the solid support and first surface 84.

Solid support 82, first surface 84, and/or first binding partner 44 may be contacted, indicated at 106, with a sample 42, which may be termed an assay solution. Contacting support 82, surface 84, and/or partner 44 with a sample may be performed by any suitable motion of the sample (and/or portions thereof), the support/surface/partner, or both.

Sample 42 may be a fluid sample. In exemplary embodiments, the fluid sample is a liquid sample, which may be aqueous. The sample also or alternatively may be described as an assay solution. The sample may contain, or may have added to it at any suitable time, second binding partner 46, second FRET member 50 (which forms a donor-acceptor FRET pair with first FRET member 48), an analyte 110, a carrier fluid 112 (e.g., water), other reagents, or any combination thereof.

Second binding partner 46 and second FRET member 50 may be connected (e.g., conjugated) to one another, covalently or noncovalently, before or after the step of contacting, to form a labeled binding member 113. In some embodiments, the analyte may be, or form a part of, the labeled binding partner. In any event, the sample may be disposed in contact with first surface 84 and/or first binding partner 44 as a pre-formed mixture or may be formed or modified by combining two or more sample components in space 104, such as by dispensing two or more fluid aliquots into space 104. In some embodiments, a fluid sample may be modified by adding labeled binding member 113 before or after the sample is contacted with the first binding partner.

To simplify the presentation here and elsewhere in the present disclosure, first FRET member 48 is shown as an energy donor and second FRET member as an energy acceptor. However, first FRET member 48 may be an energy acceptor of a FRET pair and second FRET member may be an energy donor of the FRET pair, in any of the FRET systems disclosed herein.

A binding complex 114 may be formed, indicated at 116. The binding complex may be connected to first surface 84 and solid support 82 via first binding partner 44 and/or second binding partner 46 and may include second FRET member 50.

A FRET response 118 (also or alternatively termed a FRET signal) may be measured, indicated at 120. Measurement of the FRET response may include exposing or illuminating, indicated generally at 122, first surface 84, first FRET member 48, and/or binding complex 114 to excitation light 62. Exposure may be conducted at least generally opposite sample 42, that is, with excitation light 62 reaching first surface 84 and first FRET member 48 by travel through solid support 82 toward first surface 84 (instead of through sample 42). For example, first surface 84/first FRET member 48 may be illuminated/excited with excitation light 62 via second surface 86. Exposure to excitation light may be performed with a continuous light source or with a flash light source that provides a flash (also termed a pulse) of excitation light. In any event, exposure to excitation light may induce FRET 52.

Measurement of the FRET response also may include detecting, indicated at 124, emitted light 64 passing through and/or received from the solid support in a direction away from first surface 84, such as from second surface 86 of the solid support. Detection of emitted light may be performed at any suitable time relative to exposure to excitation light, namely, at the same time, or at a distinct and/or nonoverlapping time/time interval. In some embodiments, detection of emitted light may be performed with a delay after exposure to excitation light, particularly a flash of excitation light, to provide time-resolved FRET (TR-FRET), also termed time-gated FRET. The delay may be selected to minimize detection of emission from other components in the sample, such as small organic molecules with a relatively short fluorescence lifetime (e.g., less than about 50 or 100 nanoseconds). Moreover, the energy donor of the FRET pair may have a relatively long fluorescence lifetime (e.g., greater than 100 nanoseconds, or greater than 1, 10, or 100 microseconds, among others). Accordingly, by using a delay of greater than about 50 or 100 nanoseconds, signal contribution from the energy donor and/or acceptor may be selectively enhanced relative to background signal from the other sample components that have a short fluorescence lifetime.

Fluorescence lifetime is defined as the average time a fluorophore molecule spends in the excited state before returning to the ground state and emitting a photon in the process. The typical lifetime for most fluorophores is less than 10 nanoseconds and the measurement of fluorescence is typically done in a “prompt” mode, in which the excitation and measurement of fluorescence emission take place simultaneously. Although photon emission can be measured with high sensitivity, the overall sensitivity of fluorescence detection is limited by the background light signal generated by the scattering of excitation light, as well as autofluorescence produced by the sample container and other substances in the assay solution. For fluorophores with longer fluorescence lifetimes, the sensitivity of detection can be significantly improved using a “time-gated” detection mode. In this detection mode, a “flash” light source is used to excite the fluorophore and the measurement of emission starts only after the prompt background light signal dies down. The lifetime for some lanthanide chelates is in the range of milliseconds, and this property makes them suitable labels in FRET binding assays disclosed herein.

Detection of emitted light may include any suitable detection operations. For example, detection may include detection of emitted light from the donor, detection of emitted light from the acceptor, or both, among others. Accordingly, detection may include detecting emitted light separately at two or more wavelengths (and/or ranges of wavelengths), which may be performed using filters.

The FRET response/signal may be correlated, indicated at 126, with an amount of an analyte in the sample, such as an unlabeled analyte. Correlating may involve a direct or an inverse correlation between the FRET response/signal and the amount of the analyte. Inverse correlation may, for example, be suitable in a competition assay in which the analyte interferes with formation of the binding complex by competing with a second binding partner for a limited number of binding sites. In particular, when analyte interferes in this manner, an increased amount of analyte results in a decreased interaction between the first and second binding partners, less recruitment of the second FRET member into the binding complex, and thus less energy transfer between donor and acceptor. Furthermore, correlating may be performed by a machine, such as processor (e.g., a computer) and/or by a person. Accordingly, correlating may involve use of an algorithm or a look-up table, comparison with a threshold, mental processing, or a combination thereof, among others. In some embodiments, such as in a library screen, the amount of the analyte may be correlated with an activity (also termed a characteristic) of one or more members of a library, such as a compound library. The activity may include an ability of the library member(s) to affect (inhibit/stimulate) a chemical reaction, such as an enzyme reaction, and/or an ability of the library member(s) to affect (e.g., inhibit/enhance) a binding interaction.

### III. CONNECTION OF ASSAY COMPONENTS TO A SOLID SUPPORT

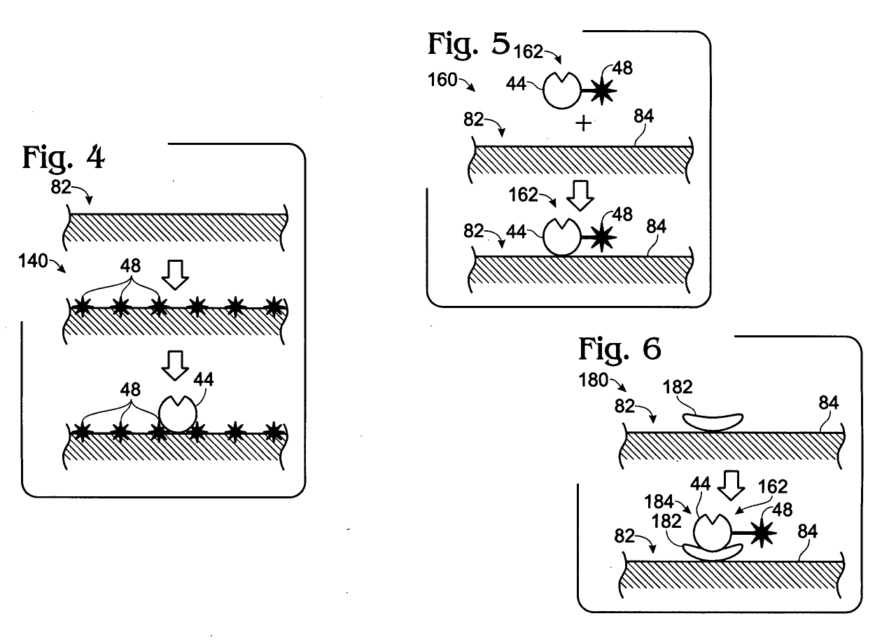

Assay components may be connected covalently or noncovalently to a solid support by any suitable linkage(s). Exemplary mechanisms for connecting assay components, particularly first binding partner 44 and first FRET member 48, are disclosed below in this section, in relation to FIGS. 4-7, and elsewhere in the present disclosure, such as in Section VII. The connection mechanisms disclosed herein and elsewhere in the present disclosure may be combined in any suitable manner to connect any suitable assay component to a solid support, at any suitable time, such as before or after the solid support is contacted with a sample.

FIG. 4 shows a schematic flowchart 140 illustrating connection of first binding partner 44 and first FRET member 48 to solid support 82. Connection may be performed sequentially, with first FRET member 48 (or first binding partner 44) connected to the solid support first, followed by subsequent connection of first binding partner 44 (or a first FRET member 48) to the solid support. The first binding partner and the first FRET member thus may have independent distributions, at the molecular level, as shown here.

FIG. 5 shows another schematic flowchart 160 illustrating connection of first binding partner 44 and first FRET member 48 to solid support 82. First binding partner 44 and first FRET member 48 may be connected to one another, for example, covalently or by noncovalent binding to form labeled partner 162, before the binding partner and first FRET member are connected to the solid support. The first binding partner and the first FRET member thus may have corresponding distributions at the molecular level.

FIG. 6 shows yet another schematic flowchart 180 illustrating connection of labeled partner 162 to solid support 82. Labeled partner 162 may be connected to solid support 82 via another binding partner 182 that is connected to solid support 82 before labeled partner 162. Binding partner 182 may bind specifically to first binding partner 44 (and/or first FRET member 48) to form a surface-bound complex 184 labeled with first FRET member 48. Accordingly, first binding partner 44 and first FRET member 48 may have corresponding distributions at the molecular level. Alternatively, the first binding partner and the first FRET member may be connected to first surface 84 independently using a distinct binding partner for each, or one of the first binding partner and the first FRET member may be connected to the solid support covalently, by entrapment, or via nonspecific interactions. Further aspects of the use of one or more additional binding partners to connect a first binding partner and/or a first FRET member to a solid support are presented elsewhere in the present disclosure, such as in Section VII (e.g., Example 10).

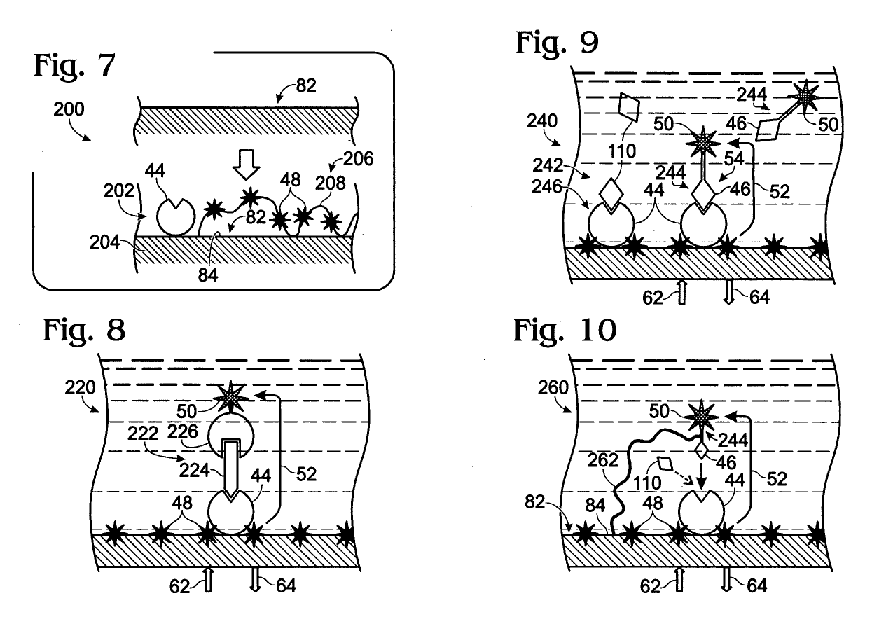

FIG. 7 shows still another schematic flowchart 200 illustrating connection of first binding partner 44 and first FRET member 48 to solid support 82. The first binding partner and the first FRET member may be disposed in a layer 202 connected to a body 204 of solid support 82, generally above first surface 84 of the solid support. Layer 202 may provide a matrix 206 (also termed a scaffold) that covalently links at least one of the first binding partner and/or the first FRET member to the matrix (e.g., see Examples 12 and 13). The matrix also or alternatively may physically entrap the first binding partner and/or the first FRET member (e.g., see Example 7) and/or may noncovalently link at least one of the first binding partner and the first FRET member to the matrix by one or more binding partner interactions, among others. Matrix 206 may be a polymer and/or may be formed of linear (substantially unbranched) molecules/chains, which may or may not be cross-linked to one another above the first surface of the solid support, or may be formed by branched molecule(s), among others. The matrix may be disposed on the solid support and particularly the first surface thereof, such that the matrix is disposed at least generally above the first surface. Furthermore, matrix 206 may be covalently connected to first surface 84 (e.g., see Examples 12 and 13) or may be noncovalently connected by binding partner interactions or by nonspecific interactions. Here, matrix 206 is formed substantially by molecules of a polymer chain 208 that is covalently connected to first FRET member 48 and to first surface 84 at a plurality of sites. First binding partner 44 may be connected to first surface 84 independently of matrix 206 and/or first FRET member 48, as shown here, or may be connected to first surface 84 via matrix 206. Independent connection of the first binding partner and the first FRET member to first surface 84 may, for example, permit use of different connection conditions (e.g., distinct solvents) for the FRET member and the binding partner, to improve connection efficiency. On the other hand, dependent connection of the first binding partner and the first FRET member may, for example, provide more positional correspondence between the binding partner and the FRET member, to increase the FRET signal, and/or may streamline the procedure for connection to the solid support, among others.

Layer 202 may have any other suitable properties. For example, layer 202 and/or matrix 206 may have any suitable thickness, such as less than about 10 or 100 nanometers, or greater than about one micrometer, among others. Matrix 206 may be porous enough to provide access of sample components, particularly a second binding partner and/or a second FRET member, to the interior of the matrix.

The polymer used to form matrix 206 may have any other suitable structure. The polymer may be a substantially linear chain, that is, a chain with few or no branches and/or little or no crosslinking between chains, such as a dextran or a maleic anhydride copolymer. The chain may have any suitable length, such as at least about 50 nanometers.

In exemplary embodiments, a surface of the solid support may be modified with a fluorophore and a binding partner disposed in a polymer matrix. For example, the binding partner and fluorophore may be attached to a long-chain polymer, and the polymer then tethered to the surface. The polymer chain may have multiple reactive groups, to allow multiple molecules of the fluorophore and binding partner to be attached to each polymer chain. This forms a three-dimensional polymer matrix on the solid surface, and each polymer chain may have multiple contacts with the surface. In a FRET binding assay, a second binding partner labeled with a second fluorophore may diffuse into the polymer matrix and form a binding complex with the binding partner already attached to the polymer chain. This brings the second fluorophore into close proximity with the fluorophore already attached to the polymer chain and results in an increase in the FRET signal. The FRET signal may be detected from the other side of the solid support. Other noncompetitive and competitive configurations of the assay based on the same principle also may be performed as described elsewhere in the present disclosure.

Matrix 206 may offer a number of substantial advantages. (1) Matrix 206 may permit more binding partner molecules to be connected to the surface of the solid support, thereby increasing the binding capacity of the surface. (2) Matrix 206 may increase the efficiency of FRET because energy donor and acceptor molecules can be brought into closer proximity in a three-dimensional matrix than with a substantially two-dimensional coating/layer on a surface. (3) Labeling and coating processes may be simplified. In particular, the polymer of matrix 206 may serve as a scaffold that supports a binding partner and/or a FRET member and that can be attached to a solid support in a single step (e.g., see Example 12).

### IV. ASSAY CONFIGURATIONS

The FRET assays disclosed herein may have any suitable configuration for detecting of one or more analytes in a sample. In practice, analytes are often not labeled. Accordingly, the presence of analyte molecules in the assay solutions may be measured using variations of the assay configuration shown in FIG. 2. Exemplary assay configurations with an unlabeled analyte are disclosed below in this section, in relation to FIGS. 8-10.

FIG. 8 illustrates an exemplary FRET system 220 that includes a binding complex 222 formed with an unlabeled analyte 224 in a non-competitive “sandwich” configuration in which binding members do not interact directly. More particularly, analyte 224 may bind to both first binding partner 44 and another binding member 226 to form a ternary complex. Binding partner 226 may be connected to second FRET member 50, such that the ternary complex positions the second FRET member proximate to first FRET member 48 for FRET 52.

FIG. 9 illustrates an exemplary FRET system 240 based on competition using an unlabeled analyte. More specifically, the system includes a sample 242 containing an unlabeled analyte 110 and a labeled analogue 244 (of analyte 110) that includes second FRET member 50. In some embodiments, the analogue may be described as a labeled competitor and/or a labeled version of analyte 110. Generally, the amount of the labeled analogue in the sample may be known/predefined and the amount of the analyte may be unknown and determined by the assay. Both analyte 110 and analogue 244 may be capable of binding to first binding partner 44. Accordingly, analyte 110 can competitively inhibit formation of binding complex 54, by preventing binding of analogue 244 through formation of an alternative binding complex 246 that does not promote FRET 52. Therefore, the FRET signal may be correlated inversely with an amount of analyte 110 in the sample. In other embodiments, the competition may be between a surface-bound analyte (or analyte analog) and analyte in solution (i.e., free analyte) (e.g., see Example 4).

The exemplary assay configurations presented in FIGS. 8 and 9 illustrate ways in which formation of a binding complex may be affected by an analyte, if any, in a fluid sample being tested for an amount and/or an activity of the analyte. In particular, the analyte may promote (FIG. 8) or interfere with (inhibit) (FIG. 9) formation of the binding complex. If the analyte promotes formation of the binding complex, a FRET response may be correlated directly with an amount of the analyte, and if the analyte inhibits formation of the binding complex, a FRET response may be correlated inversely with an amount of the analyte.

FIG. 10 illustrates an exemplary FRET system 260 based on competition, generally as described above for FIG. 9. However, in system 260, analogue 244 (i.e., second binding partner 46 and second FRET member 50) may be tethered to first surface 84 of solid support 82 via a linker 262. The linker may be flexible enough to permit motion of analogue 244, and particularly second FRET member 50, into close proximity with first surface 84 for binding of second binding partner 46 to first binding partner 44, to permit FRET 52. The linker also may be flexible and long enough to permit motion of analogue 244 away from first surface 84 and first FRET member 48, to reduce or eliminate FRET, when analogue 110 forms alternative binding complex 246 (see FIG. 9). FRET system 260 may be provided in association with any suitable solid support 82. For example, FRET system 260 may include an optical fiber as a solid support (e.g., see Section V).

Linker 262 may have any suitable composition. For example, the linker may be a long-chained polymer with hydrophilic properties, and thus may include or be formed at least mostly of polyethylene glycol, an oligonucleotide, or an oligopeptide, among others.

### V. SOLID SUPPORTS

The FRET binding assays disclosed herein are performed with a solid support. The solid support may be any solid phase member (a) to which components of a FRET binding assay can be connected covalently and/or noncovalently and (b) that allows fluorescence excitation and emission light to pass with substantial efficiency (i.e., the solid support is substantially transparent to the excitation and emission light).

The solid support may have any suitable shape, size, and composition. The solid support may be a thin plate of material (which also or alternatively may be described as a film or sheet of material), for example, with a thickness of about 0.01 to 2 or 5 millimeters, among others, or may be a continuous bulk material that is not plate-shaped. In some embodiments, the solid support may be, or may be included in, a microplate and/or a well of a microplate, with a bottom wall of the well more particularly serving as the solid support. In other embodiments, the solid support may be an optical fiber that allows excitation and emission light to pass through the fiber, generally between opposing ends thereof. The solid support may be formed of any suitable material, such as a polymeric material (e.g., polystyrene, polyacrylic, polycycloolefin, etc.), glass, or a combination thereof, among others. The solid support may be monolithic or formed of two or more discrete parts, such as a body with one or more discrete layers disposed on the body.

The solid support may have any suitable surface(s) for receiving and/or supporting a sample. The surface(s) may be substantially planar, concave, or convex, among others. Accordingly, the solid support may be or may be included in a vessel that defines a cavity for receiving a fluid sample. The cavity may be open on at least one side/end (e.g., a well), may be open on opposing sides/ends (e.g., a tube), or may be at least substantially enclosed to form a chamber (e.g., a covered/closed well and/or a sealed/capped tube).

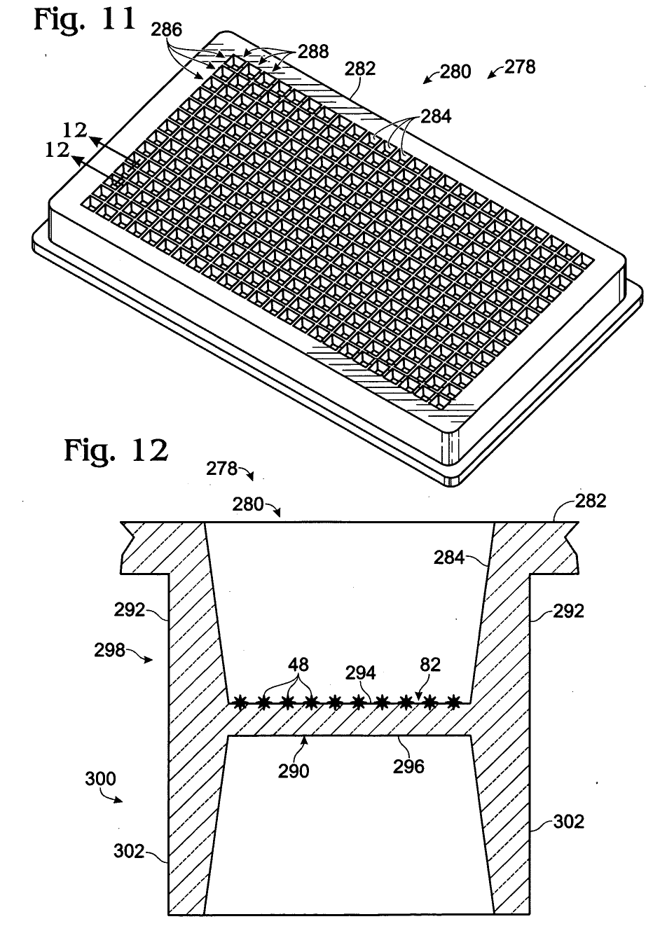

Exemplary solid supports may include and/or may be included in microplates, microscope slides, test tubes, capillary tubes, fibers, beads, or the like. Further aspects of exemplary solid supports are presented in FIGS. 11-15.

For applications in clinical diagnostics, biological sciences, and high throughput screening, most biochemical assays are performed in microplates. Microplates are typically made of plastic materials comprising a plurality of blind holes (wells). Although the number of wells on each microplate varies based on the application (e.g., 6-well microplates for some cell culture work, 3456-well microplates for some high throughput screening applications), the most common formats are 96-well and 384-well formats. For high throughput applications, 1536-well microplates are also used. Currently, most microplates used in biochemical assays have a flat bottom, regardless of the material and density of the microplates. Some more recent microplates have a V-shaped bottom that may be optimal for fluorescence detection for use in a detection system in which fluorescence is measured from above the microplate. However, in the FRET assays disclosed herein, measurement of FRET signals (illumination and detection) may take place underneath the microplates. Accordingly, it may be desirable to provide a microplate design that may be better suited for this application.

FIG. 11 shows an exemplary device, a vessel 278 structured as a microplate 280, for use as a solid support in a system for performing surface-based FRET binding assays. Microplate 280 may include a frame 282 and a plurality of wells 284 disposed in the frame and configured to hold fluid samples received from above the wells. The frame may be rectangular or may have any other suitable shape. The wells may be disposed in an at least substantially coplanar configuration and/or may be disposed in one or more rows 286 (of two more wells each) and one or more columns 288 (of two or more wells each), with the rows and columns defining respective sets of axes that are orthogonal to one another. Also, the microplate may have any suitable number of wells, such as 6, 24, 96, 392, 1536, 3456, or the like. Each well may have any suitable shape defined parallel to a plane defined by the microplate, such as square, circular, elongate (e.g., rectangular or oval), or the like. The microplate may include a cover that is received on the frame to form a top wall above each well, which may protect well contents and/or restrict evaporation of fluid from the wells. The overall dimension of the microplate and the spacing of the wells may be the same as a conventional microplate, such as those following the standard set by the Society for Biomolecular Screening.

FIG. 12 shows a cross-sectional view of a well 284 of microplate 280. Well 284 may include a bottom wall 290 and side walls 292, which may form a closed perimeter around the bottom wall to contain liquid in the well. The bottom wall may be generally planar, as shown here.

Bottom wall 290 may function as a solid support. In particular, an upper (or top) surface 294 of the well may function as a first surface 84 and a lower (or bottom) surface 296 of the well may function as second surface 86 (also see FIG. 2), and FRET signals may be measured from below the microplate/wells. The bottom wall thus may be substantially transparent and/or formed of a material that is substantially transparent. In addition, first FRET member 48 may be pre-connected to the upper surface, before a sample is placed into the well. Alternatively, or in addition, a first binding partner may be pre-connected to the upper surface. The first FRET member and/or the first binding partner may be pre-connected using any of the approaches disclosed herein, such as in Sections I, III, or VII, among others. For example, the first FRET member may be connected covalently or noncovalently to the bottom wall of the microplate, such as by entrapment in the bottom wall or via connection to a distinct polymer matrix connected to the bottom wall. The polymer matrix generally has a composition distinct from that of the bottom wall.

Any suitable type and amount of first FRET member may be pre-connected to the microplate. For example, a lanthanide, such as a lanthanide chelate and/or an inorganic lanthanide thin film, may be pre-connected to wells of the microplate. About the same amount of the first FRET member may be pre-connected to each well to provide a reproducible or similar environment in each well for fluorescence resonance energy transfer binding assays of samples added subsequently. In other words, the similar environment in each well permits comparison of assay results among wells. The microplate thus may have about the same amount of the first FRET member connected to the bottom wall of wells in each row and column and/or connected to at least substantially every well of the microplate, such as at least about 80% or 90% of the wells by number. At least some of the wells may have different amounts of a first FRET member connected to the wells, such as one or more wells with no first FRET member (as a negative control) and/or a series of wells with distinct amounts of a first FRET member, such as to measure the linearity (or non-linearity) of the FRET response. In some embodiments, a plurality of microplates may be provided, with a similar amount of a lanthanide connected to wells of each microplate, to permit comparison of assay results obtained from distinct microplates.

Well 284 may include an upper section 298 connected to a lower section 300. Upper section 298 may include bottom wall 290 and (upper) side walls 292, while lower section 300 may be formed by lower side walls 302 that depend from the bottom wall and the upper side walls. The lower side walls may or may not form a closed perimeter around the bottom wall. Also, lower side walls 302 may be substantially transparent or opaque. Furthermore, the upper side walls and the lower side walls may extend about the same distance respectively above and below the bottom wall or may extend different distances. In either case, the well may be at least generally H-shaped in vertical cross section through the center of the bottom wall, as shown here. In other embodiments, the lower side walls may not be present in the microplate.

Compared with a conventional plate design, an H-shaped microplate well may offer several advantages for fluorescence detection with optics from underneath the well. (1) Cross-talk between wells may be eliminated. With a conventional plate design, fluorescence emission light collected below one well may come from adjacent wells or from surfaces between the wells that are not in contact with the sample solution. This additional background may reduce the sensitivity of detection and may cause false assay results. (2) The bottom wall of the well that is in contact with the assay solution may be protected better from physical damage. Since excitation light and emission light may need to pass through the bottom wall of the well, any scratches or smears on the lower surface of the bottom wall may affect the signals detected. An elevated bottom provided by an H-shaped microplate well avoids contact with the lower surface of the bottom wall. (3) Enhanced focusing of fluorescence excitation light offered by the lower section of the microplate well may help to increase detection sensitivity.

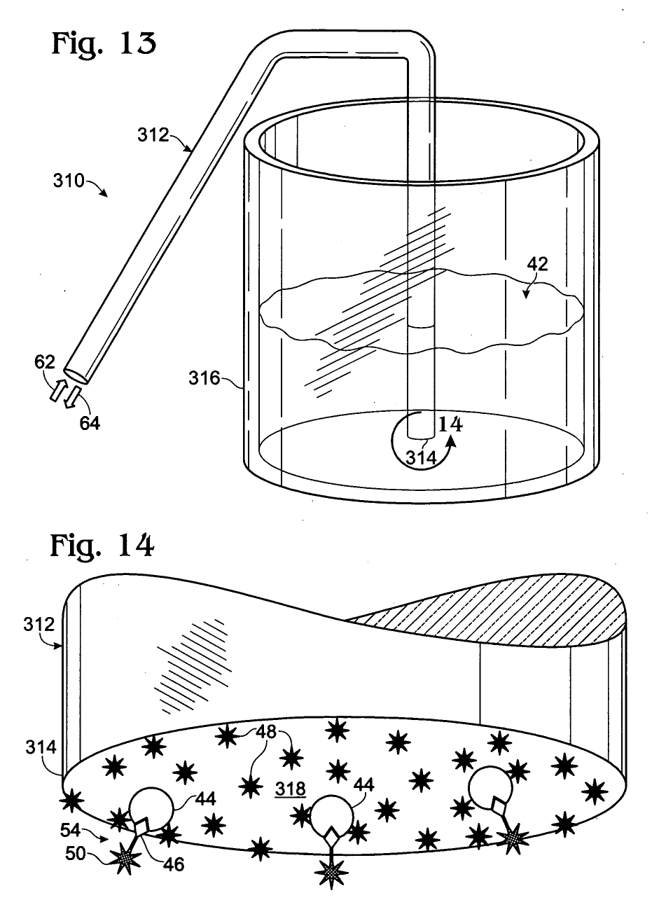

FIG. 13 shows selected aspects of an exemplary system 310 for performing surface-based FRET binding assays using an optical fiber 312 as a solid support. In FIG. 13, an end 314 of optical fiber 312 is disposed in contact with a fluid sample 42 (an assay solution) contained by a vessel 316. Excitation light 62 and emission light 64 are sent to and received from end 314 through the optical fiber, which is capable of conducting this light, to measure FRET at the end of the fiber.

FIG. 14 shows a schematic view of end 314 of optical fiber 312. The optical fiber may function as a solid support that provides an end surface 318 corresponding to first surface 84 of FIG. 2. Accordingly, first binding partner 44 and first FRET members 48 may be pre-connected to end surface 318 of the optical fiber. Contact with an assay solution containing second binding partner 46 and second FRET member 50 permits formation of binding complex 54 that increases FRET. The optical fiber may have any configuration of FRET member(s) and binding partner(s) disclosed herein. For example, the optical fiber may have a second binding partner and/or second FRET member tethered to end surface 318 of the optical fiber via a linker (e.g., see FIG. 10).

### VI. KITS

The system disclosed herein may provide a kit for performing surface-based FRET binding assays. The kit may include at least one solid support that includes at least one surface connected to a first FRET member, generally, pre-connected before contact with a fluid sample. The solid support may be, or may be included in, any of the solid supports disclosed herein, such as a vessel (e.g., a microplate). The first FRET member may be any of the FRET members disclosed herein, such as a fluorophore (donor or acceptor) or a quencher, which may be connected by any of the mechanisms disclosed herein. In exemplary embodiments, the connected first FRET member includes a lanthanide, which may be connected to a plurality of wells of a microplate. The solid support also may be pre-connected to a first binding partner. Alternatively, the kit may include a separate first binding partner that is connectable to the surface of the solid support by contacting the solid support with the first binding partner. The kit also or alternatively may include a separate second binding partner connected to a second FRET member or that binds to the second FRET member when the second binding partner and the second FRET member are mixed.

### VII. EXAMPLES

The following examples describe selected aspects and embodiments of the present disclosure, particularly methods of preparing a solid support and of performing surface-based FRET binding assays, and solid supports for surface-based FRET binding assays. These examples are included for illustration and are not intended to define or limit the entire scope of the present disclosure. The features and aspects in each of the following examples may be combined, as desired or appropriate, in any suitable manner with features and aspects of other examples and/or described elsewhere in the present disclosure.

### Example 1

### Entrapment of a Fluorophore in a Microplate

This Example describes an exemplary method of forming a microplate having a member of a FRET pair entrapped by the bottom wall of microplate wells to provide a modified upper surface of the bottom wall in each well for FRET binding assays; see FIG. 15. More particularly, this example demonstrates the feasibility of using a lanthanide chelate solution formed in an organic solvent for well treatment in the preparation of a microplate, or other plastic solid support, with a modified microplate surface that contains a member of a FRET pair, such as a fluorophore or a quencher.

### A. Materials

Microplates used are as follows: Corning 96-well clear acrylic plates with half area (catalog number 3679).

Reagents used are as follows: 1-(2-naphthoyl)-3,3,3-trifluoroacetone (NTA) from Sigma-Aldrich (catalog number 343633), and europium chloride hexahydrate (EuCl₃.H₂O) from Sigma-Aldrich (catalog number 212881).

The instrument for fluorescence detection used in the Examples of Section VII is as follows: Analyst HT plate reader from LJL Biosystems (Serial No. AN0155R).

### B. Methods

Solution A is prepared by adding 50 mg of NTA and 50 mg of EuCl₃.H₂O to 5 mL DMSO (dimethylsulfoxide) and mixing thoroughly until fully dissolved. Solution A is diluted 10-fold in DMSO to make solution B. Solution B (20 μL) then is placed into each well of a 96-well clear acrylic microplate. PBS (phosphate buffered saline; 130 μL) then is added to each well after 0, 5, 10, 20, 30, 40, 50, or 60 minutes. For time 0, PBS is added before solution B. The microplate wells then are washed three times with 150 μL/well PBS, and soaked in 150 μL/well PBS after the wash.

For fluorescence measurement, bottom optics of the Analyst plate reader are used in a “bottom read mode,” in which both the excitation light and the emission light pass through the clear bottom wall of the microplate. More specifically, excitation light reaches the microplate from below the microplate and emission light is collected from below the bottom of the microplate. The excitation filter is 360 nm and the emission filter is 620 nm. A UV dichroic filter also is used. For the long lifetime of europium chelate fluorescence, a “time resolved fluorescence” detection mode is used. In particular, emitted light is collected for 400 microseconds after a 100-microsecond delay period.

### C. Results and Discussion

The microplate is made of an organic polymer (i.e., plastic) and the surface of the plate may be modified with an organic solvent in which the polymer material is soluble. When in contact with the organic solvent, the surface may become softened and swollen, which may allow molecules such as fluorescent dyes to enter into the matrix formed by the polymer, and then become entrapped in the polymer. This technique has been used to “dye” latex microparticles. In the present study, entrapment of the fluorescent europium chelate (or other FRET member) may, in some embodiments, provide the additional benefit of protecting the chelate from an aqueous environment of a fluid sample, as water molecules are strong quenchers of the fluorescence from lanthanide chelates. The europium/NTA chelate, though highly fluorescent in DMSO, generally loses most of its ability to fluoresce when in aqueous solution. The method disclosed herein of preparing a modified surface with a physically entrapped FRET member also may have the advantage of controlling the depth of the FRET member relative to the modified surface. Conceptually, if the distance between the FRET member and the top of the surface is more than the Förster distance for fluorescence resonance energy transfer (typically 50-100 Angstroms), the FRET member generally will not contribute significantly to energy transfer and thus will only increase the background of FRET detection. By controlling the type of solvent and the duration of the solvent treatment, the depth of dye entrapment may be optimized.

After treating the plate surface with europium/NTA solution in DMSO, and washing, the microplate is soaked in aqueous solution. At this point, fluorescence detected from the microplate is principally from the treated surface, as fluorescence from free europium/NTA chelate is in contact with, and thus quenched by, water molecules. As shown in FIG. 15, there is an increase in fluorescence signal corresponding to the time of solvent treatment. About 50% or more of the fluorescence signal remains after soaking the plate for twelve hours in PBS and washing the plate three times with PBS.

### Example 2

### FRET Assays with an Entrapped Fluorophore

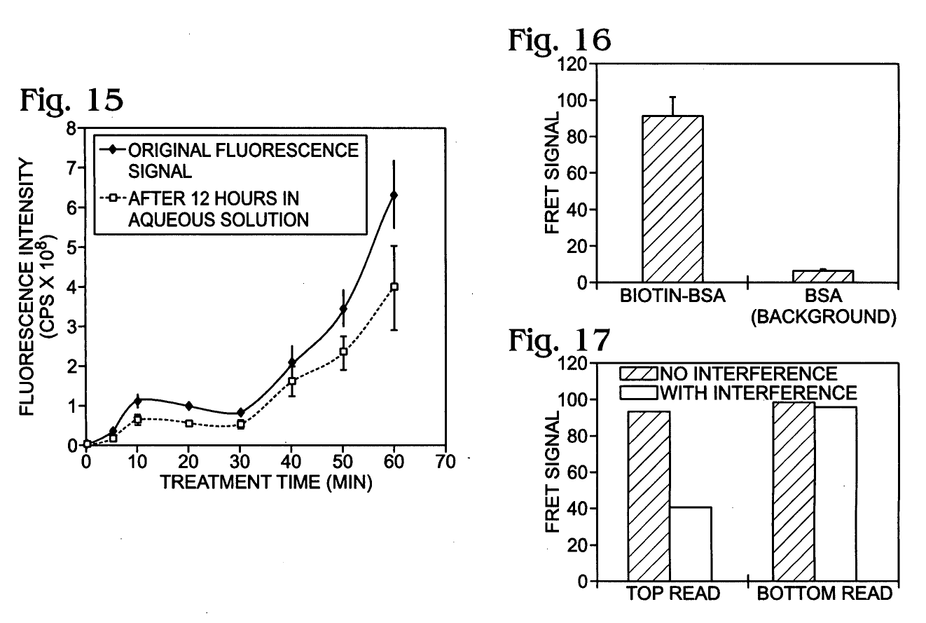

This Example describes exemplary FRET binding assays performed with modified microplates generated according to the procedure of Example 1; see FIGS. 16 and 17. One or more of the potential advantages of the novel assay technology disclosed herein are also demonstrated, such as an ability to reduce optical interference from an exemplary colored compound, namely, phenol red, disposed in samples. Phenol red is a commonly used pH indicator for cell culture and thus may occur frequently in biological samples.

### A. Materials

Microplates used are as follows: Corning 96-well clear acrylic plates are treated with europium chelate according to Example 1, to produce modified microplates with wells having a surface modified by a fluorophore (the europium chelate).

Reagents used are as follows: bovine serum albumin (BSA) and biotin-labeled bovine serum albumin (biotin-BSA) are obtained from Sigma, and allophycocyanin-labeled streptavidin (streptavidin-APC) is obtained from Perkin-Elmer.

### B. Methods

Solutions (1% (w/v)) of biotin-BSA and BSA are prepared in PBS. A 30 μL aliquot of the 1% biotin-BSA solution or the 1% BSA solution (as negative control) then is added to each well of the modified microplate. After solution addition, the microplate is incubated at room temperature for 60 minutes to allow adsorption of biotin-BSA or BSA to each well surface. The microplate then is washed three times with 150 μL/well of PBS and is soaked in 150 μL/well of PBS for two hours before the PBS solution is aspirated. To each well of the microplate, 40 μL of 50 nM streptavidin-APC solution in PBS (based on a nominal molecular weight of 400 kilodaltons) is added. The microplate then is incubated for another 60 minutes before measurement of FRET signals.

FRET measurement is performed with the Analyst plate reader with a 330 nm excitation filter with a 70 nm bandwidth and with a UV dichroic. Donor (europium) fluorescence emission is measured with a 615 nm filter with a 7.5 nm bandwidth, and acceptor (APC) fluorescence emission is measured with a 665 nm filter with a 7.5 nm bandwidth. Integration time is 800 microseconds with a 200 microsecond delay after flash excitation. Both “top read” and “bottom read” modes are used. FRET signals are calculated as 1000 times the ratio of acceptor emission to donor emission: 1000*(emission at 665 nm/emission at 615 nm). For simulated interference, 50 μL of cell culture medium including phenol red dye is added to each well.

### C. Results and Discussion

The most commonly used FRET assays in high-throughput screening for drug discovery are based on a system with a europium chelate as the fluorescence donor and allophycocyanin as the fluorescence acceptor. These assays are performed using time-resolved, fluorescence resonance energy transfer (TR-FRET) and in a homogeneous assay format in which all of the assay components are in the solution phase. In the experiments of the present Example, the feasibility of a surface-based FRET assay is tested, as well as the potential advantages of this novel assay technology.

Biotin-BSA is adsorbed to microplate well surfaces treated with a fluorescent europium chelate. The binding of streptavidin-APC to the surfaces brings the donor and acceptor dyes into proximity and a FRET signal is observed (FIG. 16). In FIG. 16, the background control is BSA with no biotin, which thus cannot bind to streptavidin-APC. The error bars in FIG. 16 represent the standard deviation of the data. Average and standard deviation values are calculated based on the results from three replicate wells.

To simulate the interference of fluorescence by light-absorbing (i.e., colored) chemicals in a typical homogeneous FRET assay, cell culture media with phenol red dye is added to some of the microplate wells (“with interference”) and left out of others (“no interference”). In a homogeneous FRET assay, both the excitation light and the emission light need to pass through a substantial portion of the assay solution, which potentially results in interference of FRET measurement due to light-absorbing substances in the assay solution. This situation is simulated using a “top read” setting on the Analyst plate reader, in the surface-based FRET binding assays disclosed herein. The “top read” setting requires excitation and emission light each to pass through a substantial portion of the assay solution, and is compared to a “bottom read” setting on the plate reader, which does not require either the excitation or emission light to substantially enter the assay solution. Average values are calculated based on the results from two replicate wells. The top read setting results in a significant decrease in the FRET signal in the presence of phenol red (FIG. 17; compare “with interference” and “no interference”). On the other hand, the new approach of the present disclosure permits excitation and emission light to pass through the bottom wall of the plate, to reach a FRET pair sequestered from the bulk assay solution by connection to the upper surface of the bottom wall. With this approach, the phenol red in the solution phase has no significant effect on the fluorescence measurement taking place at the solid phase surface (FIG. 17, “bottom read”).

In conclusion, this Example demonstrates both the feasibility and advantages of this novel assay technology compared to a conventional homogeneous TR-FRET assay.

### Example 3

### Incorporation of a Fluorophore into a Polystyrene Microplate

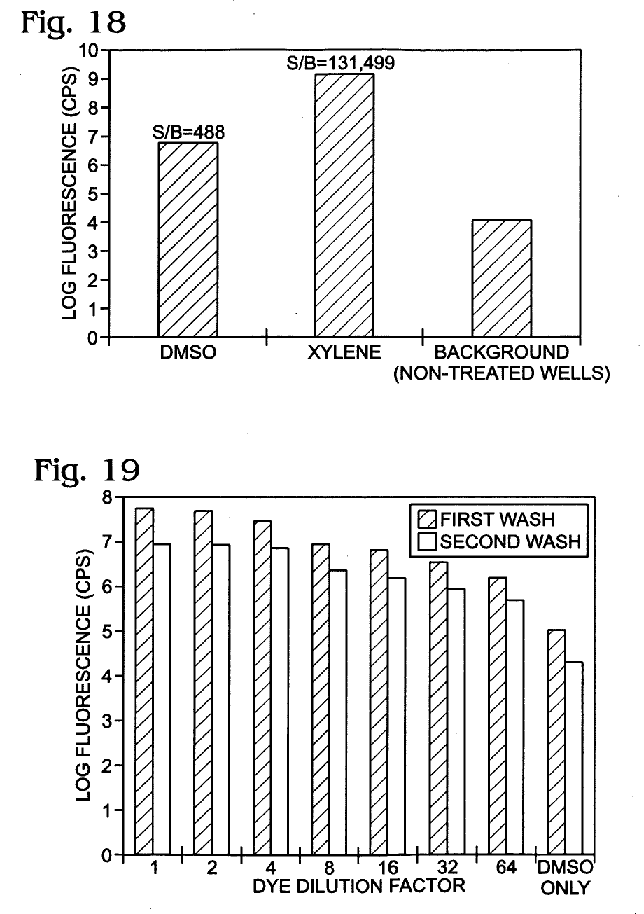

This Example describes exemplary methods of forming a microplate having a member of a FRET pair entrapped by the bottom wall of wells of the microplate, to provide a modified upper surface on the bottom wall in each well for FRET binding assays; see FIG. 18. More particularly, this Example demonstrates the feasibility of using organic solvent treatment in the preparation of a polystyrene microplate with a modified plate surface that contains a member of a FRET pair, such as a fluorophore or a quencher. However, the use of an organic solvent to connect a FRET member to a microplate surface may be applied to any suitable solid support formed of plastic or other solvent-sensitive material.

Examples 1 and 2 use acrylic-based plates to demonstrate the feasibility of preparing microplates with fluorescent surfaces for the FRET assays disclosed herein. However, the microplates commonly used in biochemical assays may be constructed of polystyrene. In this Example, a fluorescent europium chelate is used to treat polystyrene microplates, with DMSO or xylene as the solvent for the chelate.

### A. Materials

Microplates used are as follows: Corning black polystyrene 384-well microplates with clear bottoms (catalog number 3712). Reagents are as described above in Example 1.

### B. Methods

Solution A, described in Example 1, is diluted 1:5 in DMSO to make solution B and 1:5 in xylene to make solution C. An aliquot (10 μL) of solution B or solution C is added to each microplate well and incubated at room temperature for 15 minutes. For solution C, since DMSO is not soluble in xylene, two layers are formed. Solution C thus is vortexed before adding aliquots thereof to wells. In any event, after incubation in each well, the well is washed once with 80 μL DMSO and three times with 80 μL PBS. Each well next is soaked in 80 μL PBS at room temperature for three days. Each well then is washed three times with 80 μL PBS and soaked in 80 μL PBS. The europium fluorescence intensity then is measured with the Analyst HT plate reader using the settings described in Example 1.

### C. Results and Discussion

The solvent used in surface treatment of a microplate or other solid support for fluorescent dye incorporation may be an important factor in determining the efficiency of incorporation and the property of the surface. DMSO may be a good solvent for the treatment of acrylic-based polymer surfaces, as demonstrated in Example 1. However, for polystyrene microplates commonly used in biochemical assays, DMSO may not be as efficient, despite the limited solubility of polystyrene in DMSO. Therefore, a different solvent may be needed for the treatment of polystyrene surfaces to promote surface modification with a fluorophore or a quencher.

FIG. 18 shows a bar graph of the fluorescence intensity measured, plotted on a logarithmic scale. Signal to background ratios (S/B) of treated wells are also shown. Background is measured from non-treated wells. Average values are calculated based on the results from triplicate wells. In these experiments, xylene is found to be more efficient in the treatment of polystyrene surfaces for the incorporation of a fluorescent europium chelate. Furthermore, the fluorescence intensity from xylene-treated surfaces is observed to be over 100,000 times the background signal (S/B=131,499) and over 200 times higher than the signal to background ratio (S/B=488) from DMSO treatment.

### Example 4

### Dye Dilution and Competitive Binding Assays

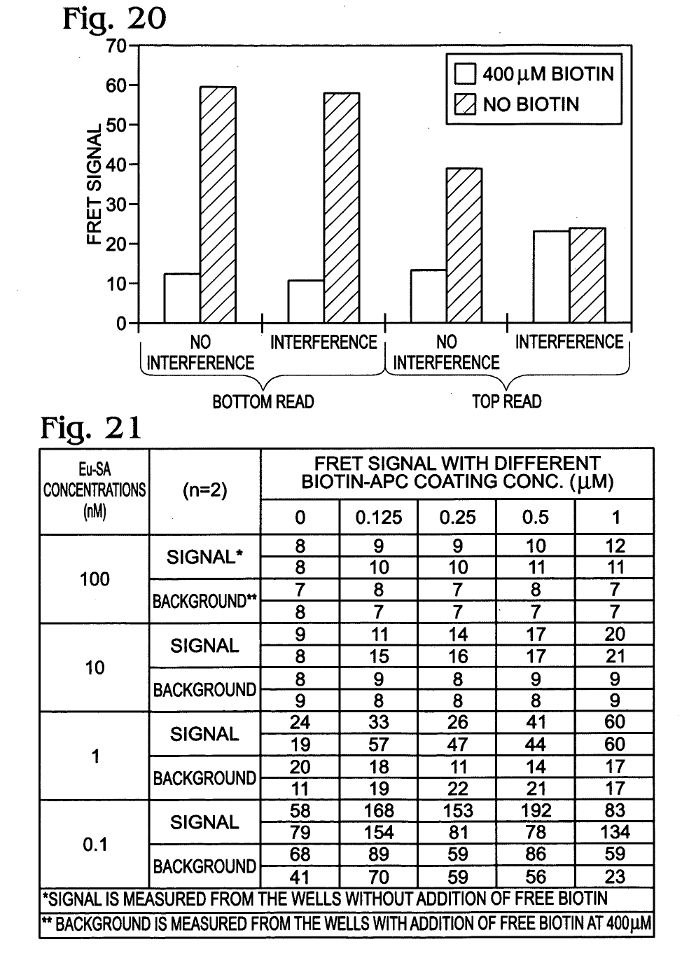

This Example describes experiments testing (a) the effect of dye dilution on microplate surface modification and fluorescence intensity thereof, and (b) FRET assays with competitive binding and simulated interference from a colored compound, phenol red; see FIGS. 19 and 20.

Examples 1 and 3 investigate time dependence (Example 1) and solvent effects (Example 3) in the solvent treatment of polymeric surfaces for the incorporation of small-molecule dyes. In this Example, the effects of dye-loading concentration are examined with polystyrene microplates and DMSO treatment. A model assay similar to that used in Example 2 is developed to further demonstrate the feasibility and potential advantages of the novel assay technology disclosed herein.

### A. Materials

Microplates used are as follows: black polystyrene 384-well microplates with clear bottoms (catalog number 3712). Reagents are as described in Examples 1 and 2.

### B. Methods

Solution A is prepared as in Example 1. Two-fold serial dilutions of solution A are prepared in DMSO, and 40 μL of each serial dilution is placed into a well of a 384-well microplate. DMSO serves as a background control. The microplate then is incubated at room temperature for 12 hours. Next, the solution is removed from each well and the well washed once with 80 μL DMSO, followed by three times with 80 μL PBS. Each well then is soaked in 80 μL PBS. Next, europium fluorescence intensity is measured with the Analyst HT plate reader using the settings described in Example 1. After incubation of each well at room temperature for a further three hours, each well is washed again with 80 μL PBS and soaked in 80 μL PBS. The europium fluorescence intensity is measured again after the second wash.

The wells treated with undiluted solution A, which had the highest fluorescence intensity, are used in a biochemical binding assay for biotin. To these wells, 20 μL of a 1% (w/v) biotin-BSA solution in PBS is added and the microplate incubated at room temperature for one hour, to bind the biotin-BSA to well surfaces (“surface-bound biotin”). Each well then is washed three times with 80 μL PBS and the final PBS solution is removed. To each well of the microplate, 10 μL of PBS with or without 400 μM biotin (“free biotin”) is added, followed by 10 μL of 200 nM streptavidin-APC in PBS. The microplate then is incubated at room temperature before FRET signals are measured. FRET measurement is performed as described in Example 2. For simulated interference, 20 μL of cell culture medium with phenol red dye is added to each well.

### C. Results and Discussion

FIG. 19 shows a bar graph of results for treatment of wells of a polystyrene microplate with DMSO solutions containing different concentrations of a Eu-NTA dye, with fluorescence intensity plotted on a logarithmic scale. Average values are calculated based on the results from sixteen replicate wells. The results provide a direct correlation between observed fluorescence intensity from a treated microplate surface and the concentration of dye loading. Compared to the previous results presented for acrylic plates (Example 1), Eu-NTA dye incorporated into a polystyrene surface is less stable (˜80% decrease in fluorescence intensity after wash). However, the remaining fluorescence intensity for the wells treated with undiluted solution A is still over 400-fold higher than background and these wells are used subsequently in the development of a competitive binding assay for biotin.

FIG. 20 shows a bar graph of FRET signals measured from streptavidin to biotin binding assays read both from the top and the bottom of the wells, and performed with and without an exemplary colored compound, phenol red, in the assay solution, as described in the Methods subsection. Average values are calculated based on the results from duplicate wells. The presence of analyte, free biotin, competes with a surface-bound biotin (biotin-BSA) for a limited number of binding sites, and results in a corresponding reduction of detected signal. As shown in FIG. 20, the presence of 400 μM biotin is detected with the assay from both top and bottom read settings in the absence of interference by a colored chemical (phenol red) in the assay solution. When the colored chemical, phenol red, is added to the assay solution, the detection of 400 μM biotin is abolished when the FRET signals are measured with “top read” optics. In contrast, the effect of interference on the FRET signals measured with “bottom read” optics is negligible. These results further demonstrate the advantage of this new assay technology over conventional homogeneous FRET assays.

### Example 5

### Surface-Based Fret Assays with a Pre-Connected Energy Acceptor

This Example describes surface-based FRET assays in which an energy acceptor of a FRET pair is pre-connected to a solid support before the solid support is contacted with a sample; see FIGS. 21 and 22.

In previous Examples, an energy donor of a FRET pair is pre-connected to a solid support to prepare a fluorescent surface on a solid support for a FRET assay. An alternative approach is to connect an energy acceptor of a FRET pair to a solid support. One of the goals of this Example is to demonstrate the feasibility of this approach by noncovalently pre-connecting an acceptor dye, allophycocyanin (APC), by adsorption to microplate well surfaces, to form labeled microplate wells, and then performing surface-based FRET assays with the labeled wells.

### A. Materials

Microplates used are as follows: Corning 384-well black microplate with clear bottom (catalog number 3711). Reagents used are as follows: biotin-labeled allophycocyanin (biotin-APC) from Prozyme (catalog number PJ25B) and europium-labeled streptavidin (Eu-SA) from Perkin-Elmer (LANCE reagents).

### B. Methods

Lyophilized, commercial biotin-APC is dissolved in PBS at 1, 0.5, 0.25, and 0.125 μM concentrations (based on a nominal molecular weight of 400 kD) and 10 μL of each solution is added to wells of a 384-well microplate. The microplate then is incubated at room temperature for one hour, followed by washing each well three times with 80 μL PBS. Next, the PBS solution in each well is removed and 20 μL of an Eu-SA solution in PBS is added to each well. The Eu-SA concentrations used are as follows: 100, 10, 1, and 0.1 nM. As a background control, 400 μM biotin is added to the Eu-SA solution prior to addition to wells of the microplate.

The plate is incubated at room temperature before FRET signals are measured as described in Example 2 using only the bottom read setting.

### C. Results and Discussion

FRET signals are commonly expressed as a ratio of acceptor emission intensity to donor emission intensity. The raw values of FRET signals therefore may be dependent on instrument settings, experimental conditions, and the concentration of the energy donor. When FRET data is interpreted, it may be advantageous to compare test signals with control signals. In this Example, control signals are obtained from wells to which 400 μM free biotin is added, which blocks the specific binding of Eu-SA to biotin-APC on the well surface.

FIG. 21 shows a table of FRET signals measured in binding assays performed with different concentrations of biotin-APC for surface-modification of microplate wells and in the presence of different concentrations of Eu-streptavidin in the assay solution. When the Eu-SA concentration is at or below 10 nM, FRET signals can be detected easily over the background signal, regardless of the absolute value of the background, which is correlated inversely with the Eu-SA concentration. With 100 nM Eu-SA, a FRET signal above background control is not detected, as the relative amount of connected biotin-APC is much lower than the Eu-SA concentration. In particular, the fluorescence emission of the donor fluorophore (europium) is so high that the emission of the acceptor fluorophore (APC) becomes undetectable.

FIG. 22 shows a bar graph of FRET signals measured in binding assays performed in microplate wells with 1 nM Eu-streptavidin in the assay solution and with the different concentrations of biotin-APC for modification of microplate wells used in FIG. 21, as described in the Methods subsection.

In summary, this experiment demonstrates the feasibility of an exemplary alternative format of the FRET assays disclosed herein, in which the acceptor of the fluorescence resonance energy transfer (FRET) pair is connected to the surface of a solid support.

### Example 6

### Fret Assays with a Pre-Connected, Small Organic Dye

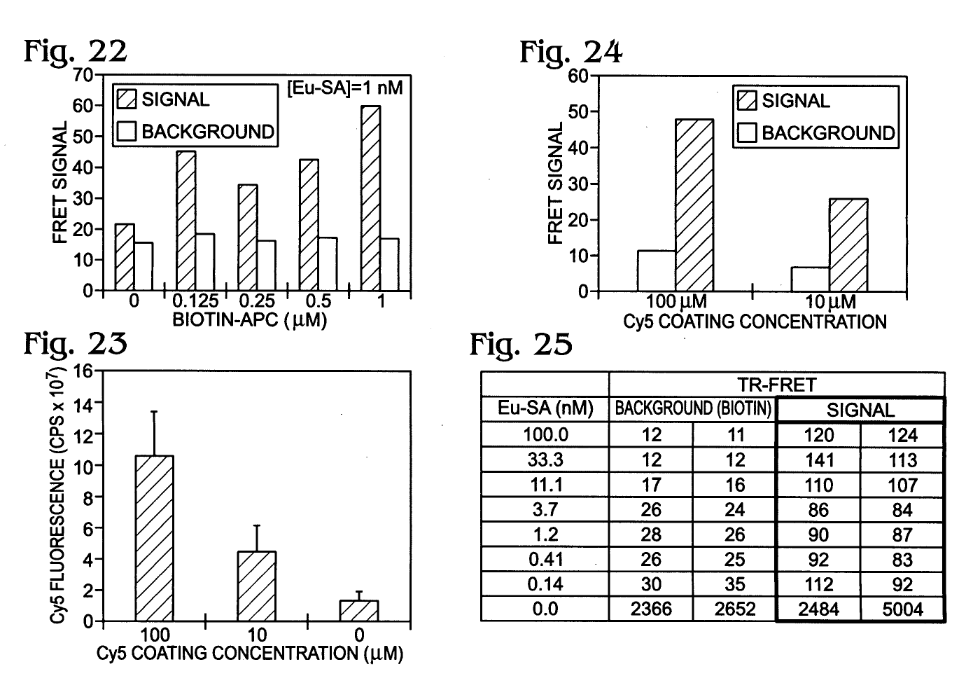

This Example describes exemplary FRET assays with a pre-connected, small organic dye, namely, Cy5; see FIGS. 23 and 24.

With europium as the donor fluorophore in time-resolved, fluorescence resonance energy transfer assays, there are other acceptor dyes that may be suitable, besides allophycocyanin (APC). In this Example, Cy5 is used as the acceptor dye and is pre-connected to a microplate surface in exemplary FRET assays.

### A. Materials

The following microplate is used: a 384-well clear acrylic UV plate (Corning catalog number 3675). Reagents used are as follows: Cy5-NHS ester (Amersham catalog number PA15101), biotin and biotin-labeled bovine serum albumin (biotin-BSA) from Sigma, and europium-labeled streptavidin (Eu-SA) from Perkin-Elmer (LANCE reagents).

### B. Methods

To each well of the 384-well plate, 20 μL of 100 μM or 10 μM Cy5-NHS in DMSO is added. The plate is incubated at room temperature for 30 minutes, followed by three washes with 80 μL PBS per well. Next, 20 μL of 0.1% (w/v) biotin-BSA solution in PBS is added to each well of the microplate and the microplate is incubated at 4° C. for three days. The plate is washed three times with 80 μL PBS per well after the incubation.

Ten μL PBS (or 100 μM biotin in PBS) is added to each well of the plate, followed by 10 μL of 4 nM Eu-SA in PBS. The microplate is then incubated at room temperature for 60 minutes before the Cy5 fluorescence intensity and FRET signals are measured with the Analyst plate reader. For measurement of Cy5 fluorescence intensity, the excitation filter is 630 nm with a 50 nm bandwidth, and the emission filter is 695 nm with a 55 nm bandwidth. FRET measurement is performed as described in Example 2 using only the bottom read setting.

### C. Results and Discussion

The plate material and solvent used in this Example are similar to those used in Example 1. However, the efficiency of dye incorporation for Cy5 is much lower than that for Eu-NTA. This may be due to the excellent water solubility of the Cy5 dye and the negative charges on the dye molecule. These properties of the Cy5 dye may make it more difficult to be incorporated into the polyacrylic surface and more likely to leak out from the surface during incubation in aqueous solution.

FIG. 23 shows a bar graph of fluorescence intensity measured from wells of a 384-well microplate treated with different concentrations of a Cy5 solution in DMSO. Average values are calculated based on the results from four replicate wells. The intensity of Cy5 fluorescence from the wells is dependent on the loading concentration of the dye, and at a Cy5 loading concentration of 100 μM, the fluorescence intensity is approximately eight-fold greater than the background with the Analyst plate reader.

FIG. 24 shows a bar graph of FRET signals measured from wells of a 384-well microplate treated as in FIG. 23 to produce Cy5-modified well surfaces, further modified with biotin-BSA to produce biotin-modified well surfaces, and then incubated with a solution of Eu-streptavidin as described in the Methods subsection. Background is measured from wells with addition of 100 μM of biotin, which blocks the binding of Eu-streptavidin to the biotin-BSA on the surface treated with Cy5. Average values are calculated based on the results from two replicate wells. The results in FIG. 24 show that FRET signals are detected over background for both 100 μM and 10 μM dye loading concentrations after adsorption of biotin-BSA to the microplate surface and addition of Eu-streptavidin. Therefore, it is still possible to develop an assay for biotin even with the relatively low concentration of Cy5 dye incorporated into the microplate surface.

### Example 7

### FRET Assays with a FRET Member Entrapped in a Matrix

This Example describes FRET binding assays performed with a FRET member disposed in a matrix or layer disposed on a solid support and physically entrapping the FRET member; see FIG. 25.

In previous Examples, a fluorescent dye used in FRET binding assays is connected to surfaces of a solid support by either noncovalent adsorption or by embedding a dye in the support matrix at and/or below the support surface using an organic solvent. The purpose of this Example includes demonstration of the feasibility of coating a solid phase surface with a polymeric material that contains a fluorescent dye. Specifically, gelatin may be used as a carrier matrix for connecting allophycocyanin (APC) dye to a microplate surface with the APC dye disposed above the surface.

### A. Materials

The following microplate is used: a 384-well clear acrylic UV plate (Corning catalog number 3675). Reagents used are as follows: biotin-labeled allophycocyanin (biotin-APC) from Prozyme (catalog number PJ25B), europium-labeled streptavidin (Eu-SA) from Perkin-Elmer (LANCE reagents), and gelatin (Knox) from a grocery store.

### B. Methods

A 1% gelatin solution is prepared by adding 0.18 g of Knox brand gelatin to 18 mL of PBS. The solution is heated to 70° C. for 30 minutes to fully dissolve the gelatin. Ten μL of 5 μM biotin-APC in PBS (based on a nominal molecular weight of 400 kD) is added to 1 mL of the 1% gelatin solution. A 10 μL volume of the gelatin solution is then added to each well of a 384-well microplate. The microplate is shaken briefly and stored at room temperature for 12 hours to allow the gelatin to solidify and form a matrix containing biotin-APC.

The microplate is washed three times with 100 μL PBS. Next, 20 μL of PBS (or 20 μL of 100 μM biotin in PBS as a background control) is added to each well of the microplate, followed by 20 μL of a Eu-SA solution in PBS at a concentration of 100, 33, 11, 3.7, 1.2, 0.41, 0.14 or 0 nM Eu-SA. The microplate is incubated at room temperature for 120 minutes before FRET signals are measured with the Analyst plate reader as described for Example 2 using only the bottom read mode.

### C. Results and Discussion

FRET signals are commonly expressed as a ratio of acceptor emission intensity to donor emission intensity. FRET signals therefore may be dependent on the instrument settings, experiment conditions, and concentration of the fluorescence donor. When FRET data is interpreted, it may be advantageous to compare test FRET values with FRET values obtained from controls that provide a measure of the background FRET signal. In the present Example, background controls are created by addition of 100 μM (free) biotin to the wells, which blocks binding of solution phase Eu-SA to biotin-APC in the gelatin matrix.

FIG. 25 is a table of FRET signals measured from wells of a 384-well microplate treated with a gelatin solution of biotin-APC to connect the biotin-APC to a surface of the wells, via a gelatin matrix, before contact with different concentrations of Eu-streptavidin, as described in the Methods subsection. Background controls are measured from wells containing 100 μM biotin, which blocks the binding of Eu-streptavidin to the biotin-APC. FRET signals can be detected easily over the background signal at each Eu-SA concentration tested, regardless of the absolute value of the background, which is correlated inversely with the Eu-SA concentration. At the highest Eu-SA concentration tested, 100 nM, the signal to background ratio is greater than 10 fold.

### Example 8

### FRET Assays with a Lanthanide Film

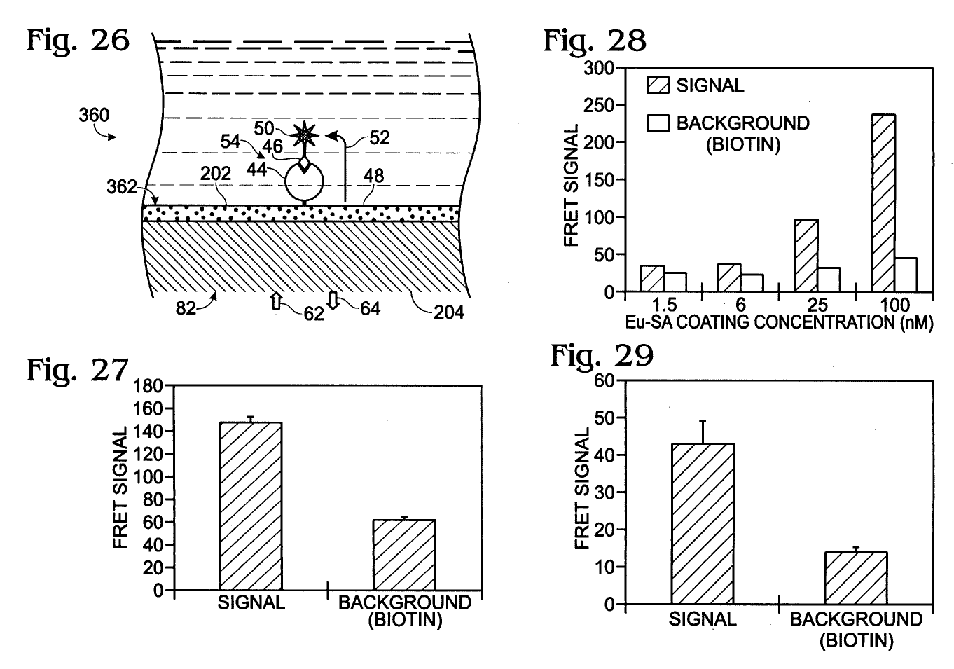

This Example describes the use of a thin film of inorganic lanthanide in FRET binding assays; see FIG. 26.

Commercialized TR-FRET assays include the HTRF™ system from Cisbio (www.htrf.com), LANCE™ system from Perkin Elmer, and LanthaScreen™ system from Invitrogen. At the center of these TR-FRET systems are proprietary fluorescent lanthanide chelates. Finding a suitable chelate for TR-FRET applications is a challenging task, as the fluorescence of lanthanide ions is quenched by water molecules and most of the fluorescent chelates are not stable in aqueous solutions, especially at the low concentrations needed for the assay. In the HTRF system, a cryptate is used to protect the europium ion from water molecules. However, even with the most advanced chemistry developed over the last two decades, the fluorescence quantum yield of the lanthanide chelate is typically below 0.1 in aqueous solutions. In contrast, the quantum yields of inorganic lanthanide phosphors in a solid phase may be close to 1.

One of the goals of the present disclosure is to apply some of the material technologies developed in electronics and semiconductor industries to the development of novel surface-based TR-FRET assays. Over the last four decades, a vast amount of work has been directed towards the development of lanthanide phosphors for color TV cathode ray tubes and fluorescent lamps. Lanthanide phosphors with different properties are readily available commercially. Vapor deposition technologies are also available at an industry scale for the coating of these phosphors onto substrate surfaces. Finally, new developments in plasma coating technologies allow easy activation of these surfaces for the attachment of biomolecules.

These technologies from the electronics and semiconductor industries may be combined to produce a sensing surface formed above a thin film of inorganic lanthanide. By optimizing vapor deposition conditions, the thickness of the thin film may be controlled to be less than about 10 or 20 nm, with 10 nm being the range of the typical Förster distance for TR-FRET. It may be advantageous to limit the thickness of the phosphor coating because phosphor molecules that are at a distance much greater than the Förster distance from the film surface will have low efficiency for TR-FRET, while potentially contributing to fluorescence background, which may reduce the sensitivity of FRET detection. A phosphor thin film disposed under a layer, such as a monolayer of coating through plasma deposition of silane material, may separate the phosphor from a sample, such as an aqueous assay solution. The coating/layer over the phosphor thin film also may introduce one or more functional groups for the covalent attachment of biomolecules, such as a binding partner. Since all of the technologies and necessary equipment are already used in large-scale production in other industries, a sensing surface may be produced in large quantities at low per-unit cost.

FIG. 26 shows selected aspects of an exemplary system 360 for performing FRET binding assays using an inorganic lanthanide thin film 362. Thin film 362 may be formed as a layer 202 on solid support 82, such as on a body 204 of the solid support, with the body and/or solid support being substantially transparent. The thin film may serve as first FRET member 48, particularly, an energy donor for second FRET member 50 of the FRET pair, which serves as an energy acceptor for FRET 52. First binding partner 44 may be connected to thin film 362, such that the first binding partner is at least substantially immobilized with respect to the thin film and accessible to a sample or assay solution disposed adjacent the solid support. The connection of first binding partner 44 to thin film 362 may be covalent or noncovalent. Binding of second binding partner 46 to first binding partner 44 may result in formation of binding complex 54, which increases FRET 52 measured via excitation light 62 and emission light 64 that travel to and from the binding complex through solid support 82.

### Example 9

### Fret Assays with a Lanthanide Pre-Connected in Aqueous Solution

This Example describes FRET binding assays that use a first FRET member, such as a lanthanide chelate, that is pre-connected to a surface of a solid support in aqueous solution before contacting the surface with sample; see FIG. 27. The purpose of this Example includes demonstration of the feasibility of using treatment with an aqueous, fluorescent europium chelate solution in the preparation of microplates with modified microplate surfaces that contain a fluorescent material, and the application of the microplate surfaces in FRET assays.

### A. Materials

The following microplate is used: 384-well small volume microplate with non-binding surface treatment (Corning catalog number 3544). Reagents used are as follows: 4′,4′-Bis(4,4,5,5,6,6,6-heptafluoro-1,3-dioxohexyl)-o-terphenyl-4′-sulfonyl chloride (BHHCT) is from Sigma-Aldrich (catalog number 59752), europium chloride hexahydrate (EuCl₃.H₂O) is from Sigma-Aldrich (catalog number 212881), biotin-labeled bovine serum albumin (biotin-BSA) is from Sigma, and allophycocyanin-labeled streptavidin (streptavidin-APC(SA-APC)) is from Perkin-Elmer.

### B. Methods

Add 50 μL of 10 mM BHHCT in DMF (dimethylformamide) and 5 μL of 100 mM EuCl₃.H₂O in water into 445 μL of PBS (phosphate buffered saline) and mix thoroughly until fully dissolved to make solution C. Add 10 μL solution C into each well of the microplate and incubate at room temperature for 45 minutes. Each well of the microplate then is washed three times with 50 μL PBS. Next, 10 μL of 33 μM biotin-BSA in PBS is added to each well and incubated at room temperature for 30 minutes. Each well of the microplate is then washed three times with 50 μL PBS. Finally, 10 μL of 100 nM SA-APC in PBS is added to each well. For control wells, 10 μL of 100 nM SA-APC, 2 μM biotin in PBS is added. The wells are then incubated for another 60 minutes before measurement of FRET signals with the Analyst plate reader as described above for Example 2 except that only a bottom read mode is used and the FRET signals are calculated as 10000 times the ratio of acceptor emission to donor emission:

FRET=10000*(emission at 665 nm/emission at 615 nm).

### C. Results and Discussion

The procedure of this Example uses fluorescent europium in an aqueous solution with 10% DMF to treat a polystyrene plastic surface and results in a highly fluorescent surface. This surface is then used to develop a surface-based FRET binding assay.

FIG. 27 shows a bar graph of FRET signals measured from wells of a microplate assayed for streptavidin-allophycocyanin binding to surface-connected biotin on well surfaces modified with a europium chelate, as described in the Methods subsection. Background control is with excess free biotin to block the binding. Error bars represent the standard deviation of data from the data mean value determined from four replicate wells. The results show that a FRET signal resulting from binding of first and second binding partners is detectable without further assay optimization.

### Example 10

### FRET Assays with a Binding Partner Conjugated to an Energy Donor

This Example describes FRET binding assays performed with a binding partner conjugated to an energy donor; see FIG. 28.

The purpose of this Example includes demonstrating the feasibility of using a binding protein conjugated to a fluorescent europium chelate in the preparation of microplates with modified surfaces and the application of these modified surfaces in FRET assays.

### A. Materials

The following microplate is used: 384-well microplate coated with streptavidin (Nunc Immobilizer Streptavidin plate). The following reagents are used: biotin-BSA is from Pierce (catalog number 29130), europium-labeled streptavidin (Eu-SA) is from Perkin Elmer (catalog number AD0061), and biotin-labeled allophycocyanin (biotin-APC) is from Prozyme (catalog number PJ25B).

### B. Methods

Ten μL of 1 μM biotin-BSA in PBS is added to each well of the streptavidin coated microplate, and the microplate is incubated at room temperature for 30 minutes. The microplate is washed three times with 80 μL PBS per well. Next, 10 μL of 1.5, 6, 25 or 100 nM Eu-SA in PBS is added to unique sets of wells of the microplate. After 30 minutes of incubation at room temperature, the microplate is washed three times with 80 μL PBS per well. Finally, 10 μL of 100 nM biotin-APC in PBS is added to each well. For control wells, the 100 nM biotin-APC solution also includes 2 μM biotin. The microplate is then incubated for another 60 minutes before measurement of FRET signals without removing, diluting, or otherwise modifying the sample composition in the wells. Instrument settings for FRET measurement are as described in Example 2 and FRET signals are calculated as in Example 9.

### C. Results and Discussion

The setup of this assay is very similar to a typical “homogeneous” FRET assay, with the exception that the binding complex is formed on a microplate surface. Fluorescent europium chelate-labeled streptavidin is captured to the microplate surface, via pre-connected biotin-BSA, and then biotin-labeled APC is added to form a binding complex on the microplate surface.

FIG. 28 shows a bar graph of FRET signals measured from wells of a microplate assayed for biotin-APC binding to Eu-SA, with the Eu-SA connected to surfaces of the wells before contact with the biotin-APC as described in the Methods subsection. Background controls include excess free biotin to block the binding. Average values are calculated based on the results from duplicate wells. FRET signals can be detected in this system using a “bottom-read” fluorescence detection setup. The potential advantage of this assay compared to conventional “homogeneous” FRET assay is that the interference of fluorescence detection by colored and/or fluorescent substances in the assay solution may be reduced.

### Example 11

### FRET Assays with an Energy Donor Pre-Connected at a Surface

This Example describes FRET binding assays using an energy donor covalently attached to the surface of a solid support; see FIG. 29. One of the goals of this Example is to demonstrate the feasibility of direct chemical attachment of a fluorescent europium chelate to a microplate surface and the application of this microplate surface in FRET binding assays.

### A. Materials

The following microplate is used: 384-well small volume microplate (Corning catalog number 3540) treated with plasma to generate amino groups on the microplate surface.

Reagents used are as follows: 4′,4′-Bis(4,4,5,5,6,6,6-heptafluoro-1,3-dioxohexyl)-o-terphenyl-4′-sulfonyl chloride (BHHCT), europium chloride hexahydrate (EuCl₃.H₂O), pyridine, and nitromethane is from Sigma-Aldrich (catalog number 212881); biotin-labeled bovine serum albumin (biotin-BSA) is from Sigma; and allophycocyanin labeled streptavidin (SA-APC) is from Perkin-Elmer.

### B. Methods

Ten μL of 100 μM BHHCT in nitromethane containing 1% pyridine is placed into each well of the microplate and incubated at 4° C. overnight. The plate is washed three times with 50 μL PBS containing 0.1% Tween-20. Thirty μL of 100 μM EuCl₃.H₂O in water is added to each well and incubated at room temperature for 60 minutes. Each well is then washed again three times with 50 μL PBS, and 10 μL of 33 μM biotin-BSA in PBS is added to each well. The microplate is incubated at 4° C. overnight and then the wells are washed three times with 50 μL PBS per well. Finally, 10 μL of 100 nM SA-APC in PBS is added to each well. For control wells, 10 μL of 100 nM SA-APC in PBS with 2 μM biotin is added. The microplate is then incubated for another 60 minutes before measurement of FRET signals using instrument settings described in Example 2, with FRET signals calculated as in Example 9.

### C. Results and Discussion

The microplate is treated with plasma to generate amino groups on the surface, which allow covalent attachment of fluorescent europium chelate BHHCT through the chlorosulfonyl group. More particularly, a chelator, such as BHHCT, may be covalently linked to the microplate surface first, and then a lanthanide may be chelated with the chelator in a distinct step. Alternatively, a lanthanide chelate may be covalently linked as a pre-formed chelate. In any event, a layer (e.g., a monolayer) of fluorescent europium chelate may be connected covalently to the microplate surface.

FIG. 29 shows a bar graph of FRET signals measured from wells of a microplate assayed for streptavidin-allophycocyanin binding to biotin-BSA on well surfaces modified with a europium chelate, as described in the Methods subsection. The background control is with excess free biotin to block the binding. Error bars represent the standard deviation of the mean data value, calculated based on the results from three replicate wells. FRET signals can be detected without further optimization of the assay conditions.

### Example 12

### FRET Assays with a Matrix Attached to a Solid Support

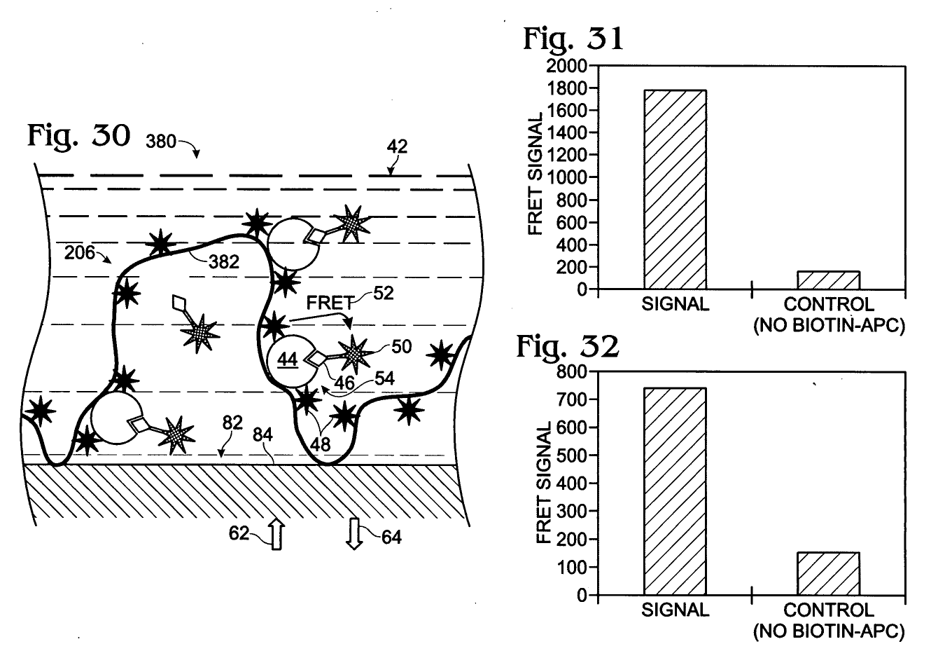

This Example describes FRET binding assays performed with a first FRET member and a first binding partner covalently linked to a solid support via a polymer matrix; see FIGS. 30 and 31.

The purpose of this experiment is to demonstrate the feasibility of using a polymer matrix coating in the preparation of microplates with a modified surface that contains fluorescent materials, and the application of this modified surface in FRET binding assays.

FIG. 30 shows an exemplary system 380 for performing surface-based FRET binding assays, with the system illustrating the general strategy followed in this Example. System 380 may include a polymer matrix 206 covalently connected to first surface 84 of solid support 82. Polymer matrix 206 may include, and/or may be formed at least substantially of, a long-chain polymer (e.g., chain(s) 382 of at least about 10 or 100 kilodaltons), to which each of first binding partner 44 and first FRET member 48 is connected, covalently or noncovalently. Matrix 206 first may be connected to first binding partner 44 and/or first FRET member 48 and then tethered to the solid support through covalent bonds (or noncovalently), or matrix 206 may be connected to solid support 82 before first binding partner 44 and/or the first FRET member 48.

The solid support including connected matrix 206, first binding partner 44, and first FRET member 48 may be contacted with sample 42 that includes second binding partner 46 connected to second FRET member 50. Formation of binding complex 54 permits increased FRET 52, which may be measured using excitation light 62 and emission light 64. Each individual energy transfer may occur from an individual first FRET member 48 disposed on the same or a different individual chain of the matrix.

In the remainder of this Example, the first binding partner is streptavidin, the second binding partner is biotin, the first FRET member is a europium chelate as an energy donor, and the second FRET member is allophycocyanin (APC) as an energy acceptor.

### A. Materials

The following microplate is used: 384-well polypropylene plate with natural (clear) color and flat bottom from Sigma (catalog number 1686). The microplate is treated with plasma to generate amine groups on the surface.

The following reagents are used: biotin-labeled allophycocyanin (biotin-APC) from Prozyme (catalog number PJ25B), streptavidin from Pierce Biotechnology, and poly(methyl vinyl ether-alt-maleic anhydride) (PMVMA) with an average molecular weight of 216 kD from Sigma (catalog number 416339).

### B. Methods

One μL of 50 μM PMVMA in DMSO is added to 50 μL of 50 μM streptavidin in PBS and incubated at room temperature for 30 minutes. The solution is then diluted 100-fold in PBS containing 30 μM fluorescent europium chelate, which contains free primary amine functional groups. Twenty μL of the diluted solution is added to each well of the polypropylene microplate, which has amino functional groups on the surface, and then the microplate is incubated at room temperature for 2.5 hours. The wells of the microplate are then washed three times with 100 μL PBST wash buffer (PBS containing 0.1% Tween-20). For the assay, 20 μL of 10 nM biotin-APC in PBST is added to each well and incubated for 1 hour at room temperature. Measurement of FRET signals is performed using the instrument settings described in Example 2, with FRET signals calculated as in Example 9.

### C. Results and Discussion

Based on average molecular weight, each PMVMA polymer chain contains over a thousand reactive cyclic anhydride functional groups. The polymer chain links the amino groups on the streptavidin, the fluorescent europium chelate, and the microplate surface to produce a three dimensional matrix coating. Binding of biotin-APC to the streptavidin brings the acceptor dye into the polymer matrix and enables fluorescence resonance energy transfer with the donor dye in the matrix.

FIG. 31 shows a bar graph of FRET signals measured from wells of a microplate assayed for biotin-allophycocyanin binding to streptavidin, as described in the Methods subsection. Average values are calculated based on the results from duplicate wells. The FRET signal increases by over 10-fold as a result of binding. Since the anhydride group also hydrolyzes in aqueous solution, the timing of the coating process may benefit from careful control to achieve reasonable coupling yields.

### Example 13

### FRET Assays with Two-Step Attachment to a Solid Support

This Example describes connecting a matrix, a first FRET member, and a first binding partner to a solid support in a two-step process in which the matrix and the first FRET member are connected to the solid support in a first step and then the first binding partner is connected to the solid support, independently of the matrix and first FRET member, in a second step; see FIG. 32.

In this Example, the coating process is divided into two steps. In a first step, a fluorescent europium chelate reacts with a maleic anhydride co-polymer in an organic solvent and the mixture is then connected covalently to a microplate surface, along with a bi-functional chemical linker. A protein solution is added in a second step for connection of protein molecules to the microplate surface. Since the first step is done in organic solvent, the coupling yield can be improved. The second step then can be performed in aqueous solution. A bi-functional linker helps to improve the efficiency of protein immobilization.

### A. Materials

The following microplate is used: 384-well polypropylene plate with natural (clear) color and flat bottom from Sigma (catalog number 1686). The microplate is treated with plasma to generate amine groups on the surface.

The following reagents are used: reagents listed in Example 12 and disuccinimidyl suberate (DSS) from Sigma.

### B. Methods

A DMSO solution with 0.1 μM PMVMA and 50 μM fluorescent europium chelate with primary amine functional groups is prepared and incubated at room temperature for 40 minutes. To this solution, a DSS solution in DMSO is added to make a mixture with 10 μM DSS. Ten μL of the mixture is added to each well of the polypropylene microplate having amino functional groups on the microplate surface. The microplate then is incubated at room temperature for 2.5 hours. The microplate is washed three times with 100 μL isopropyl alcohol and dried. For protein immobilization, 20 μL of 1.0 μM streptavidin in PBS is added to each well and the microplate is incubated at room temperature for 2 hours. After the incubation, the microplate is washed three times with 100 μL PBST. For the assay, 20 μL of 10 nM biotin-APC in PBST is added to each well and incubated for 1 hour at room temperature. Measurement of FRET signals is performed using the instrument settings described in Example 2, with FRET signals calculated as in Example 9.

### C. Results and Discussion

Use of organic solvents in the coupling of a fluorescent europium chelate to the PMVMA polymer and the attachment of the modified polymer to the microplate surface circumvents the problems associated with the instability of the cyclic anhydride functional groups on the PMVMA in aqueous solution. The two-step coating process also allows the functionalized microplates to be prepared and stored separately. Addition of DSS helps to improve the efficiency of the protein immobilization, since the N-hydroxyl succinimidyl ester moiety on DSS is relatively more stable in aqueous solution and is more efficient for coupling of protein molecules.

FIG. 32 shows a bar graph of FRET signals measured from wells of a microplate assayed for biotin-allophycocyanin binding to streptavidin generally as in FIG. 31 except with the use of the two-step coupling procedure described in the Method subsection. The FRET signal increases by five-fold as a result of binding of biotin-APC molecules to streptavidin. Average values are calculated based on the results from duplicate wells.

### Example 14

### Exemplary Embodiments

A solid support may be modified to include at least one FRET member by any suitable mechanism. Either a donor or acceptor fluorophore may be associated with a solid phase surface of the support. The fluorophore may be adsorbed noncovalently to the surface. Alternatively, or in addition, the fluorophore may be attached covalently to the surface through chemical bonding. In other examples, the fluorophore may form a complex with a binding agent (i.e., a binding partner) and the complex may be associated with the surface by a covalent or noncovalent mechanism. The fluorophore may be coated on the surface along with a polymeric material. Alternatively, the fluorophore may be embedded into a polymeric material surface. In some embodiments, the polymeric material may be treated with an organic solvent containing a fluorophore. The organic solvent may allow molecules of the fluorophore to penetrate and to be entrapped in the polymeric material near the surface. In other embodiments, a solid support may be coated with a gelatin solution containing allophycocyanin (APC). A different polymeric material containing another fluorophore may be coated onto the solid support in a similar fashion. In some examples, a fluorescent europium-BHHCT (4,4-bis(1,1,2,2,3,3-heptafluoro-4,6-hexanedion-6-yl)chlorosulfo-o-terphenyl) chelate may be covalently attached to a glass surface with a chemically modified surface (e.g., treated with a silanizing agent (such as 3-Aminoproypl trimethoxylsilane (APTMS))). In other examples, the fluorophore may be attached to a polymer such as a dextran or a maleic anhydride co-polymer and the resulting complex then may be attached to the solid surface. In further examples, the fluorophore may be an inorganic phosphor thin film coated on the solid support surface.

### Example 15

### Selected Embodiments

This Example describes selected embodiments of the present disclosure, presented as a series of numbered paragraphs.

1. A method of detecting an analyte in a fluid sample, the method comprising: (A) contacting a binding partner with a fluid sample, the binding partner being connected to a first surface of a solid support that also has a first member of a fluorescence resonance energy transfer (FRET) pair connected to the first surface; (B) forming a binding complex connected to the first surface via the binding partner and including a second member of the FRET pair, formation of the binding complex being affected by an analyte, if any, in the fluid sample; (C) exposing the FRET pair to excitation light via a second surface of the solid support, with the second surface spaced from the sample; (D) measuring a FRET response of the FRET pair to the step of exposing by detecting emitted light received from the second surface, wherein excitation light reaches the FRET pair, and emitted light is received from the FRET pair, without passing through a substantial portion of the sample, thereby minimizing optical interference from the sample; and (E) correlating the FRET response with an amount of the analyte in the sample.

2. The method of paragraph 1, wherein the step of exposing includes exposing the first surface to a flash of excitation light via the second surface, and wherein detecting includes detecting emitted light with a delay after the first surface is exposed to the flash of excitation light.

3. The method of paragraph 1 or 2, wherein the step of forming a binding complex is performed with the first member of the FRET pair entrapped by the solid support.

4. The method of paragraph 1 or 2, wherein the step of forming a binding complex is performed with the first member of the FRET pair disposed at or above the first surface.

5. The method of any one of paragraphs 1 to 4, wherein the binding partner and the first member of the FRET pair have respective molecular distributions, and wherein the step of contacting is performed with the respective molecular distributions being substantially independent of one another.

6. The method of any one of paragraphs 1 to 5, wherein the step of contacting is performed with the second member of the FRET pair already connected to the first surface through a linker including polyethylene glycol and/or an oligonucleotide.

7. The method of any one of paragraphs 1 to 6, wherein the step of contacting is performed with the first FRET member and the binding partner connected to each other only via the solid support.

8. The method of paragraph 7, wherein each of the first FRET member and the binding partner is connected covalently to the solid support.

9. The method of any one of paragraphs 1 to 8, wherein the binding partner is a first binding partner, wherein the step of forming a binding complex forms a binding complex that includes a second binding partner bound to the first binding partner

10. The method of paragraph 9, wherein the step of contacting contacts the first binding partner with a fluid sample that includes the second binding partner covalently linked or noncovalently bound to the second member of the FRET pair.

11. The method of any one of paragraphs 1 to 10, wherein the analyte interferes with formation of the binding complex, and wherein the step of correlating includes a step of correlating the FRET response inversely with an amount of the analyte in the sample.

12. The method of any one of paragraphs 1 to 11, further comprising repeating the steps of contacting, forming, exposing, measuring, and correlating with a plurality of fluid samples.

13. The method of paragraph 12, wherein each solid support is included in a distinct well of a microplate.

14. The method of paragraph 12, wherein each fluid sample includes one or more members of a library of compounds, further comprising a step of correlating the amount of the analyte in each fluid sample with an activity of the one or more members included in each fluid sample.

15. The method of paragraph 14, wherein the activity relates to whether and/or how much the one or members of a library included in each sample affect a chemical reaction performed in the sample.

16. The method of paragraph 14, wherein the activity relates to whether and/or how much the one or more members included in each sample affect a binding interaction.

17. A method of detecting an analyte in a fluid sample, the method comprising: (A) adding to a fluid sample a binding partner connected to a member of a fluorescence resonance energy transfer (FRET) pair; (B) contacting a surface of a solid support with the fluid sample, the solid support being substantially transparent and having an other member of the FRET pair substantially immobilized with respect to the surface; (C) forming a binding complex connected to the surface via the binding partner, formation of the binding complex being affected by an analyte, if any, in the fluid sample; (D) passing excitation light through the solid support towards the surface to expose the FRET pair to the excitation light; (E) measuring a FRET response of the FRET pair to exposing by detecting emitted light that has traveled through the solid support away from the surface, wherein excitation light reaches the FRET pair, and emitted light is received from the FRET pair, without passing through a substantial portion of the sample, thereby minimizing optical interference from the sample; and (F) correlating the FRET response with an amount of the analyte in the sample.

18. The method of paragraph 17, wherein the step of contacting is performed before the step of adding.

19. A device for assaying an analyte in fluid samples, comprising: (A) a microplate forming a plurality of wells for holding fluid samples and each having a bottom wall that is substantially transparent such that optical detection can be performed from below the bottom wall, the bottom wall including upper and lower surfaces; and (B) a fluorescent lanthanide connected to the upper surface of each bottom wall, with about a same amount of the fluorescent lanthanide being connected to each upper surface, thereby providing a similar environment in each of the wells for surface-based fluorescence resonance energy transfer (FRET) assay of fluid samples.

20. The device of paragraph 19, further comprising a binding partner connected to the upper surface of the bottom wall.

21. The device of paragraph 19 or 20, further comprising a polymer matrix connected to the upper surface of the bottom wall, wherein the lanthanide is connected to the upper surface of each bottom wall through the polymer matrix.

22. The device of any one of paragraphs 19-21, wherein the lanthanide is an inorganic lanthanide phosphor.

23. The device of any one of paragraphs 19-21, wherein the lanthanide includes a lanthanide chelate.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A method of detecting an analyte in a fluid sample, the method comprising:
contacting a binding partner with a fluid sample, the binding partner being connected to a first surface of a solid support that also has a first member of a fluorescence resonance energy transfer (FRET) pair connected to the first surface;
forming a binding complex connected to the first surface via the binding partner and including a second member of the FRET pair, formation of the binding complex being affected by an analyte, if any, in the sample;
exposing the FRET pair to excitation light via a second surface of the solid support, with the second surface spaced from the sample;
measuring a FRET response of the FRET pair to the step of exposing by detecting emitted light received from the second surface, wherein excitation light reaches the FRET pair, and emitted light is received from the FRET pair, without passing through a substantial portion of the sample, thereby minimizing optical interference from the sample; and
correlating the FRET response with an amount of the analyte in the sample.

2. The method of claim 1, wherein one of the first and second members of the FRET pair is an energy donor having a fluorescence lifetime of greater than about 100 nanoseconds, and wherein detecting emitted light is performed with a delay greater than about 100 nanoseconds after exposing the first surface with a flash of excitation light via the second surface, thereby selectively reducing background fluorescence, if any, from fluorophores having a fluorescence lifetime of less than the delay.

3. The method of claim 1, further comprising a polymer matrix connected to the solid support, wherein the step of forming a binding complex is performed with the first member of the FRET pair and/or the binding partner connected covalently to the first surface of the solid support through the polymer matrix.

4. The method of claim 1, further comprising a step of connecting the first member of the FRET pair to the binding partner before the first member of the FRET pair and the binding partner are connected to the first surface of the solid support.

5. The method of claim 1, wherein the step of contacting is performed with the second member of the FRET pair already connected to the first surface through a flexible linker.

6. The method of claim 1, wherein the step of measuring includes a step of measuring a FRET response in which the first member of the FRET pair functions as an energy donor and the second member of the FRET pair functions as an energy acceptor, or in which the first member of the FRET pair functions as an energy acceptor and the second member of the FRET pair functions as an energy donor.

7. The method of claim 1, wherein one of the first and second members of the FRET pair is a fluorophore and the other of the first and second members is a fluorescence quencher.

8. The method of claim 1, wherein the step of contacting is performed with first and second members of the FRET pair that are fluorophores.

9. The method of claim 1, wherein at least one of the first and second members of the FRET pair includes a lanthanide.

10. The method of claim 1, wherein one of the first and second members of the energy transfer pair is an energy donor that includes a europium (III) chelate or a terbium (III) chelate.

11. The method of claim 1, wherein one of the first and second members of the energy transfer pair is an energy acceptor that includes a phycobiliprotein.

12. The method of claim 11, wherein one of the first and second members of the energy transfer pair is an energy acceptor that includes allophycocyanin.

13. The method of claim 1, wherein the step of contacting is performed with the first FRET member being an inorganic lanthanide phosphor disposed on the solid support as a thin film.

14. The method of claim 1, wherein the step of contacting is performed with the solid support being provided by a microplate forming a plurality of wells each having a bottom wall that includes upper and lower surfaces, and wherein the first surface is the upper surface of the bottom wall of a well.

15. The method of claim 1, wherein the solid support is an optical fiber.

16. The method of claim 1, further including a step of correlating the amount of the analyte with an activity of one or more compounds in the sample, the one or more compounds being one or more members of a library of compounds being screened.

17. A method of detecting an analyte in a fluid sample, the method comprising:
adding to a fluid sample a binding partner connected to a member of a fluorescence resonance energy transfer (FRET) pair;
contacting a surface of a solid support with the sample, the solid support being substantially transparent and having an other member of the FRET pair substantially immobilized with respect to the surface;
forming a binding complex connected to the surface via the binding partner, formation of the binding complex being affected by an analyte, if any, in the sample;
passing excitation light through the solid support towards the surface to expose the FRET pair to the excitation light;
measuring a FRET response of the FRET pair to exposing by detecting emitted light that has traveled through the solid support away from the surface, wherein excitation light reaches the FRET pair, and emitted light is received from the FRET pair, without passing through a substantial portion of the sample, thereby minimizing optical interference from the sample; and
correlating the FRET response with an amount of the analyte in the sample.

18. A device for assaying an analyte in fluid samples, comprising:
a microplate forming a plurality of wells for holding fluid samples and each having a bottom wall that is substantially transparent such that optical detection can be performed from below the bottom wall, the bottom wall including upper and lower surfaces; and
a fluorescent lanthanide connected to the upper surface of each bottom wall, with about a same amount of the fluorescent lanthanide being connected to each upper surface, thereby providing a similar environment in each of the wells for surface-based fluorescence resonance energy transfer (FRET) assay of fluid samples.

19. The device of claim 18, wherein the fluorescent lanthanide is connected covalently to the upper surface of each bottom well.

20. The device of claim 18, wherein each well has an at least generally H-shaped cross-section, thereby permitting fluorescence detection to be performed below each individual well with minimal optical interference from adjacent wells.
